(19) United States
(12) Patent Application Publication
(10) Pub. No.: US 2020/0207830 A1
(43) Pub. Date: Jul. 2, 2020

(54) TREM2 MUTANTS RESISTANT TO SHEDDASE CLEAVAGE

(71) Applicant: NOVARTIS AG, Basel (CH)

(72) Inventors: Dominik FEUERBACH, Müllheim (DE); Ulf NEUMANN, Rheinfelden (CH)

(21) Appl. No.: 16/633,797

(22) PCT Filed: Jul. 26, 2018
(86) PCT No.: PCT/IB2018/055592

§ 371 Date: Jan. 24, 2020

Related U.S. Application Data
(60) Provisional application No. 62/537,486, filed on Jul. 27, 2017.

(51) Int. Cl. C07K 14/705; C12N 15/86; C12N 7/00

(52) U.S. Cl. CPC C07K 14/70503; C12N 15/86; A61K 48/00; C12N 2750/14143; C12N 7/00

(57) ABSTRACT

Provided herein are methods and compositions related to TREM2 mutants resistant to sheddase cleavage, e.g., human TREM2 mutants resistant to sheddase cleavage, and nucleic acids encoding such TREM2 mutants resistant to sheddase cleavage.

### SEQUENCE LISTING

The instant application contains a Sequence Listing which has been submitted electronically in ASCII format and is hereby incorporated by reference in its entirety. Said ASCII copy, created on Jul. 20, 2018, is named PAT057836-WO-PCT_SL.txt and is 60,538 bytes in size.

### TECHNICAL FIELD

The present invention provides methods and compositions related to TREM2 mutants resistant to sheddase cleavage, e.g., human TREM2 mutants resistant to sheddase cleavage, and nucleic acids encoding such TREM2 mutants resistant to sheddase cleavage.

### BACKGROUND

Triggering receptors expressed on myeloid cells or “TREMs” are a group of transmembrane glycoproteins that are expressed on different types of myeloid cells, such as mast cells, monocytes, macrophages, dendritic cells, and neutrophils. TREMs have an immunoglobulin (Ig)-type fold in their extracellular domain and thus belong to the immunoglobulin superfamily (IgSF). TREM receptors contain a short intracellular domain, but lack docking motifs for signaling mediators and require adapter proteins, such as DAP12 (DNAX-activating protein of 12 kDa) for cell activation. Two members of TREMs have been reported: TREM1 and TREM2, both of which play an important role in immune and inflammatory responses.

TREM2 is expressed on macrophages, dendritic cells, osteoclasts, microglia, lung epithelial cells and hepatocarcinoma cells, but absent from myeloid cells in the blood. TREM2 physically associates with DAP12, which acts as a signaling adaptor protein for TREM2 and a number of other cell surface receptors. The cytoplasmic domain of DAP12 contains an immunoreceptor tyrosine activation motif (ITAM) (Wunderlich, J. Biol. Chem. 288, 33027-33036, 2013). After activation of the interacting receptor, DAP12 undergoes phosphorylation at the two conserved ITAM tyrosine residues by Src kinases. Subsequent recruitment and activation of the Syk protein kinase trigger downstream signaling pathways, including the activation of mitogen-activated protein kinase (MAPK) and phospholipase Cγ (PLCγ).

TREM2 can be activated by lipopolysaccharides (LPS), heat shock protein 60, neuritic debris, bacteria, and a broad array of anionic and zwitterionic lipids, e.g. phosphatidic acid (PA), phosphatidylglycerol (PG), phosphatidylserine (PS), phosphatidylinositol (PI), phosphatidylcholine (PC) and sphingomyelin. TREM2 activation increases phagocytic capacity of microglia and macrophages, reduces the release of pro-inflammatory cytokines and limits TLR signaling. TREM2 sustains microglial survival by synergizing with CSF-1 receptor signaling. Further, TREM2 interacts with Plexin-A1 regulating cellular adhesion and motility. TREM2 signaling facilitates degradation of ingested prey and is crucial for lipid metabolism, myelin uptake and intracellular breakdown.

TREM2 undergoes sequential proteolytic processing by ectodomain shedding and intramembrane proteolysis (Wunderlich, J. Biol. Chem. 288, 33027-33036, 2013). During ectodomain shedding, the ectodomain of TREM2 is released by proteases such as members of the ADAM (a disintegrin and metalloproteinase domaincontaining protein) or BACE (b-site APP cleaving enzyme) family (Kleinberger, Sci Transl Med. 2014; 6(243):243ra86). After removal of the ectodomain, the remaining membrane-retained fragment is further processed by γ-secretase mediated intramembranous proteolysis. Soluble fragments of TREM2 (sTREM2) produced by ectodomain shedding have been observed in supernatants of dendritic cell cultures as well as in plasma and CSF samples from patients with noninflammatory neurological diseases and multiple sclerosis (Kleinberger, 2014). The shed ectodomain of TREM2 (sTREM2) in human CSF has been assessed as a potential Alzheimer's disease (AD) biomarker and has been shown to be increased during ageing in general (Suarez-Calvet, EMBO Molecular Medicine 8, 466-476, 2016). Detailed analysis during the course of AD revealed that sTREM2 increases early in AD before clinical symptoms appear, peaks in MCI-AD, and stays elevated but at lower levels compared to the MCI-AD stage in AD dementia (Suarez-Calvet, 2016).

### SUMMARY OF THE INVENTION

Provided herein are human TREM2 mutants resistant to sheddase cleavage (e.g., human TREM2 mutants resistant to ADAM17 or ADAM10 cleavage), nucleic acids encoding human TREM2 mutants resistant to sheddase cleavage, and vectors and cells containing such nucleic acids. Also provided herein are methods of increasing TREM2 expression in a subject and methods of treating a TREM2-related disease or disorder in a subject by using nucleic acids encoding human TREM2 mutants resistant to sheddase cleavage, or vectors and cells containing such nucleic acids.

In one aspect, provided herein are nucleic acids comprising a sequence encoding a human TREM2 mutant resistant to sheddase cleavage (e.g., human TREM2 mutant resistant to DAM17 or ADAM10 cleavage).

In some embodiments, the human TREM2 mutant comprises a stalk region comprising an amino acid sequence selected from any one of SEQ ID NOs: 33-40. In some embodiments, the human TREM2 mutant comprises a stalk region consisting of an amino acid sequence selected from any one of SEQ ID NOs: 33-40. In some embodiments, the human TREM2 mutant comprises a stalk region comprising an amino acid sequence selected from any one of SEQ ID NOs: 33-35. In some embodiments, the human TREM2 mutant comprises a stalk region consisting of an amino acid sequence selected from any one of SEQ ID NOs: 33-35. In some embodiments, the human TREM2 mutant comprises an amino acid sequence selected from any one of SEQ ID NOs: 41-48. In some embodiments, the human TREM2 mutant comprises an amino acid sequence selected from any one of SEQ ID NOs: 41-48.

In some embodiments, provided herein are nucleic acids comprising any one of SEQ ID NOs: 67-74. In some embodiments, provided herein are nucleic acids comprising any one of SEQ ID NOs: 67-69. In some embodiments, provided herein are nucleic acids comprising any one of SEQ ID NOs: 67, 70, or 74. In some embodiments, provided herein are nucleic acids comprising SEQ ID NO: 67.

In some embodiments, such nucleic acids can comprise a promoter, e.g., a constitutive promoter, an inducible promoter, a synthetic promoter, or a cell-type specific promoter. In some embodiments, the promoter is a cell-type specific promoter. For example, the promoter can drive the nucleic acid expression specifically in microglias, macrophages, or dendritic cells. In some embodiments, such nucleic acids comprise a promoter selected from a TREM2 promoter, TMEM119 promoter, Hexb promoter, IBA1 promoter, CD45 promoter, CD11b promoter, Cst7 promoter, Lpl promoter, Csf1 promoter, Cs1R promoter, Itgax promoter, Clec7a promoter, Lilrb4 promoter, Tyrobp promoter, Ctsb promoter, Ctsd promoter, B2m promoter, Lyz2 promoter, Cx3cr1 promoter, Cst3 promoter, Ctss promoter, P2ry12 promoter, C1qa promoter, or C1qb promoter. In some embodiments, such nucleic acids comprise a TREM2 promoter. In some embodiments, such nucleic acids can comprise a polyadenylation signal.

In some embodiments, the nucleic acids comprising a sequence encoding a human TREM2 mutant resistant to sheddase cleavage can further comprise a second sequence encoding a DAP12 protein. In some embodiments, such nucleic acids comprise an internal ribosome entry site upstream of the second sequence. In some embodiments, such nucleic acids comprise a 2A sequence upstream of the second sequence, e.g., a 2A sequence selected from any one of SEQ ID NOs: 52-66. The DAP12 protein can comprise SEQ ID NO: 49. In some embodiments, the DAP12 protein consists of SEQ ID NO: 49.

In another aspect, provided herein are vectors (e.g., expression vectors) that comprise nucleic acids comprising a sequence encoding a human TREM2 mutant resistant to sheddase cleavage. Such vectors can be a DNA vector, an RNA vector, a plasmid, a cosmid, or a viral vector. In some embodiments, the vector is a viral vector is selected from a vector based on any one of the following viruses: lentivirus, adenovirus, adeno-associated virus (AAV), Herpes Simplex Virus (HSV), parvovirus, retrovirus, vaccinia virus, Sinbis virus, influenza virus, reovirus, Newcastle disease virus (NDV), measles virus, vesicular stomatitis virus (VSV), poliovirus, poxvirus, Seneca Valley virus, coxsackievirus, enterovirus, myxoma virus, or maraba virus. In some embodiments, the vector is a lentiviral vector. In some embodiments, the vector is an AAV vector. In some embodiments, the vector further comprises a selectable marker.

In another aspect, provided herein are cells comprising a nucleic acid or a vector that comprise a sequence encoding a human TREM2 mutant resistant to sheddase cleavage. Such cells can be a macrophage, a dendritic cell, or a microglia. In some embodiments, the cell can expresse a detectable marker.

In a further aspect, provided herein are polypeptides that comprise an amino acid sequence selected from any one of SEQ ID NOs: 33-40. Also provided are polypeptides that comprise an amino acid sequence selected from any one of SEQ ID NOs: 41-48.

In a further aspect, provided herein are methods of increasing TREM2 expression in a subject (e.g., a human) by administering to the subject any of the nucleic acids, vectors, or cells described herein. The subject can have a TREM2-related disease or disorder. Such nucleic acids, vectors, or cells can be administered to the subject through an intravenous, intracranial, intrathecal, subcutaneous, or intranasal route. The methods can further comprise administering a second agent to the subject.

In a further aspect, provided herein are methods of treating a TREM2-related disease or disorder in a subject (e.g., a human) in need thereof, the method comprising administering to the subject any of the nucleic acids, vectors, or cells described herein. Such nucleic acids, vectors, or cells can be administered to the subject through an intravenous, intracranial, intrathecal, subcutaneous, or intranasal route. The methods can further comprise administering a second agent to the subject.

In some embodiments, the TREM2-related disease or disorder is a neuroinflammatory or neurodegenerative disease selected from Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, sphingomyelinlipidose (Niemann-Pick C), mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis. In some embodiments, the TREM2-related disease or disorder is Alzheimer's disease. In some embodiments, the TREM2-related disease or disorder is frontotemporal dementia.

In some embodiments, the methods described herein can further comprise assaying the cell surface human TREM2 level in a sample (e.g., a cerebrospinal fluid sample) obtained from a subject. The cell surface human TREM2 level in a sample can be determined by an assay selected from flow cytometry, immunohistochemistry, Western blotting, immunofluorescent assay, radioimmunoassay (RIA), enzyme-linked immunosorbent assay (ELISA), homogeneous time resolved fluorescence (HTRF), or positron emission tomography (PET).

Also included are uses of the nucleic acids, vectors, cells, or polypeptides described herein for treatment of a TREM2-related disease or disorder in a subject. Uses of the nucleic acids, vectors, cells, or polypeptides described herein in the manufacture of a medicament for treatment of a TREM2-related disease or disorder in a subject are also contemplated.

### BRIEF DESCRIPTION OF THE DRAWINGS

FIG. 1A shows exemplary alignment of the amino acid sequences of human TREM2 isoform 1 (SEQ ID NO: 1), Cyno TREM2 isoform 1 (SEQ ID NO: 5), and mouse TREM2 isoform 1 (SEQ ID NO: 6). The stalk region of TREM2 includes the light grey shaded residues. The transmembrane domain of TREM2 includes the underlined residues. FIG. 1B shows exemplary alignment of the amino acid sequences of human TREM2 isoform 1 (SEQ ID NO: 1), isoform 2 (SEQ ID NO: 3), and isoform 3 (SEQ ID NO: 4). FIG. 1C shows exemplary alignment of the amino acid sequences of the stalk regions of human TREM2 isoform 1 (SEQ ID NO: 89), isoform 2 (SEQ ID NO: 8), and isoform 3 (SEQ ID NO: 9). FIG. 1D shows exemplary alignment of the amino acid sequences of the stalk regions of human TREM2 isoform 1 (SEQ ID NO: 7), Cyno TREM2 isoform 1 (SEQ ID NO: 10), and mouse TREM2 isoform 1 (SEQ ID NO: 11). FIG. 1E illustrates the structure of TREM2 and its interaction with the signaling adaptor protein DAP12. Mature TREM2 includes a single immunoglobulin (IgSF) domain, a stalk region, a transmembrane (TM) domain, and a cytoplasmic domain.

FIGS. 2A-2E show ADAM17 is the pivotal sheddase for the cleavage of TREM2 ectodomain in CHO-hDAP12-hTREM2 cells and human M2A macrophages. FIGS. 2A-2B show TREM2 cell surface expression in CHO-hDAP12-hTREM2 after treatment with ADAM inhibitors DPC333 (black circle) or GI254023 (upward triangle); additional phorbol-myristate-acid (PMA) treatment was applied in FIG. 2B. FIGS. 2C-2D show TREM2 cell surface expression in human M2A macrophages after treatment with ADAM inhibitors DPC333 (black circle) or GI254023 (upward triangle); additional PMA treatment was applied in FIG. 2D. TREM2 cell surface staining is plotted as mean intensities corrected for nuclear staining as mean±S.E. (n=3). A representative experiment from two experiments is shown. FIG. 2E is a line graph showing inhibition of recombinant ADAM10 and ADAM17 by DPC333 and GI254023 in vitro in Hepes buffer at pH 7.5.

FIGS. 3A-3D are bar graphs showing ADAM17 but not ADAM10 TREM2 knockout THP1 cells show increased TREM2 expression and reduction of shed TREM2 (sTREM2). FIG. 3A shows TREM2 cell surface expression in THP1 CRISPR cell clones. FIG. 3B shows sTREM2 level of supernatants from THP1 C CRISPR cell clones. Data are plotted as mean±S.E. (n=2). *p<0.01, statistical difference to untreated group of Ctrl gRNA clone. #p<0.01, statistical difference to PMA treated group of Ctrl gRNA clone. CtrlgRNA is control gRNA transfected clone; AD10 H4 is ADAM10 CRISPR knockout clone; AD17 G12 is ADAM17 CRISPR knockout clone. FIG. 3C shows lack of ADAM10 expression in THP1 ADAM10 H4 CRISPR clone. Left panel control clone CtrlgRNA; right panel AD10 H4 clone. FIG. 3D is a representative Western blot analysis of THP1 control clone CtrlgRNA (lane 1) and ADAM17 AD17 G12 CRISPR cells (lane 2), showing lack of ADAM17 expression in THP1 ADAM17 G12 CISPR clone.

FIGS. 4A-4D show the amino acid stretch in the membrane proximal part of the TREM2 stalk region are important for shedding. FIG. 4A shows the amino acid sequence of the membrane proximal part of the wild type or mutant human TREM2 stalk region. Amino acid numbering according to iProt Q9NZC2. TM: transmembrane region. Gaps indicate deleted amino acids within the respective mutant. Exchanged amino acids in bold. Underlined amino acids indicates main sheddase cleavage site for generation of the C-terminus of TREM2 ectodomain. As shown in Table 4, WT: SEQ ID NO: 12; TRUNC3 (159-174 deletion): SEQ ID NO: 13; TRUNC1: SEQ ID NO: 14; T2del 3-8: SEQ ID NO: 15; T2del 6-11: SEQ ID NO: 16; T2del 11-16: SEQ ID NO: 17; T2-YGG: SEQ ID NO: 18; T2-WFR: SEQ ID NO: 19; T2-double: SEQ ID NO: 20; T2-IPD: SEQ ID NO: 21; T2-IPP: SEQ ID NO: 22, T2-IDP: SEQ ID NO: 23. FIG. 4B is a bar graph showing FACS analysis of TREM2 WT and mutants transiently expressed in HEK293-FT cells. Cells were treated 48 hours after transfection with 50 ng/ml PMA or 0.05% DMSO. Cells were detached, labelled with AF1828 antiserum and analyzed by flow cytometry. Data are plotted as the ratio of untreated over PMA treatment for each mutant as mean±S.E. (n=3). Statistical differences to WT were calculated by Anova with Dunnett's multiple comparison test. *P<0.01. FIG. 4C is a bar graph showing gene activation by TREM2-double mutant as shown in FIG. 4A. TREM2-double mutant was stably expressed in BWZ-lacZ-mDAP12 cells. Cells were seeded on an activating monoclonal antibody or isotype control and reporter gene activity assessed after 16 h. Data are plotted as the ratio of RGA for activating/control Ab as mean±S.E. (n=3). FIG. 4D is a bar graph showing replacement of the three amino acids at the sheddase cleavage site strongly increases TREM2 cell surface expression. FACS analysis of TREM2 WT and mutants transiently expressed in HEK293-FT cells. Cells were treated 48 h after transfection with 50 ng/ml PMA or 0.05% DMSO. Cells were detached, labelled with AF1828 antiserum and analyzed by flow cytometry. Data are plotted as the ratio of untreated over PMA treatment for each mutant as mean±S.E. (n=3). Statistical differences were calculated by Anova with Dunnett's multiple comparison test. *P<0.01.

FIGS. 5A-5E show ADAM17 cleaves TREM2 stalk region peptides in vitro at H157-S158. FIG. 5A shows the amino acid sequences of the TREM2 stalk region derived synthetic peptides for in vitro cleavage assays. All peptides were obtained with an N-terminal 7-methoxycoumarin (Mca) fluorescent tag at the C-terminus. Underlined amino acids indicates main sheddase cleavage site. AA112-171: SEQ ID NO: 24; Peptide 1: SEQ ID NO: 25; Peptide 2: SEQ ID NO: 26; Peptide 3: SEQ ID NO: 27; Peptide la: SEQ ID NO: 28; Peptide 2a: SEQ ID NO: 29; Peptide 4: SEQ ID NO: 30; Peptide 5: SEQ ID NO: 31; Peptide 6: SEQ ID NO: 32. FIG. 5B shows HPLC analysis of cleavage of Peptide 3 (10 μM) by ADAM17 (31 nM) for 1, 5, or 24 hours. FIG. 5C shows HPLC analysis of cleavage of Peptide 3 (10 μM) by ADAM17 (31 nM) for 48 hours, with identification of the major product and 2 minor products. FIG. 5C discloses SEQ ID NOS 83, 51 and 84, respectively, in order of appearance. FIG. 5D shows the time course of ADAM17 cleavage of Peptide 1, Peptide 2, or Peptide 3 (mean of 2 experiments). FIG. 5E shows the time course of ADAM17 cleavage of Peptide 4, Peptide 5, or Peptide 6 (mean of 2 experiments).

FIG. 6A shows identification of C-terminus of TREM2 ectodomain shed from HEK-FT cells transiently transfected with WT or R47H human TREM2 (SEQ ID NO: 85) and DAP12. Ion extracts of the 3-time charged [D137-H157] peptide ion, m/z 791.94-792.06. The peptide ion of interest is clearly present in all four shed TREM2 tryptic digests (boxed ion extract). * represents an unknown peptide present as a 5-time charged ion and ** is the deaminated form of the same peptide. FIG. 6B shows deconvoluted MS^{E} spectrum of peptide D₁₃₇-H₁₅₇ (SEQ ID NO: 85). Top panel indicates which b and y fragment ions are identified. This mass spectrum is from the tryptic digest of TREM2 R47H PMA.

FIG. 7 shows identification of shed TREM2 ectodomain from HEK-FT cells transiently transfected with WT or mutant R47H hTREM2 and hDAP12. Deconvoluted mass spectra of 4 mass spectra: the shed hTREM2 [19-157]is clearly present in all four cell supernatant extracts. The cell supernatants had been treated with PNGase-F and Sialidase A after affinity purification, but not reduced.

FIGS. 8A-8C shows determination of O-glycosylation site(s) within TREM2 stalk region. TREM2-His was first treated with Sialidase A, then reduced, alkylated, subsequently treated by PNGase-F. The resulting sample was then either digested by trypsin or by Asp- and Glu-C enzyme. The digests were analyzed by LC-MS^{E}. FIG. 8A: Deconvoluted mass spectrum, combined scans: 1926:2097 (SEQ ID NO: 86). FIG. 8B: Deconvoluted mass spectrum, combined scans: 1566:1584 (SEQ ID NO: 87). FIG. 8C: Deconvoluted mass spectrum, combined scans: 1373:1477 (SEQ ID NO: 88).

### DETAILED DESCRIPTION

Provided herein are human TREM2 mutants resistant to sheddase cleavage (e.g., human TREM2 mutants resistant to ADAM17 or ADAM10 cleavage), nucleic acids encoding human TREM2 mutants resistant to sheddase cleavage, and vectors and cells containing such nucleic acids. Also provided herein are methods of increasing TREM2 expression in a subject and methods of treating a TREM2-related disease or disorder in a subject by using nucleic acids encoding human TREM2 mutants resistant to sheddase cleavage, or vectors and cells containing such nucleic acids.

TREM-2 mediates non-phlogistic phagocytosis of bacteria and dying cells and dampens inflammatory responses. Homozygous loss of function of human TREM-2 causes Nasu-Hakola disease (polycystic lipomembranous osteodysplasia with sclerosing leukoencephalopathy, “PLOSL”), or fronto-temporal dementia (FTD)-like syndrome, diseases characterized by bone cysts, neuroinflammation, progressive neurodegeneration and presenile dementia. A heterozygous loss of function mutation R47H of TREM-2 is also an important risk factor for late-onset Alzheimer's disease (AD), with an effect size that is similar to that of the apolipoprotein E ε4 allele. TREM-2 is expressed in the microglia found in the white matter, hippocampus and neocortex, which is partly consistent with the pathological features reported in AD brains, supporting the possible involvement of TREM-2 in AD pathogenesis. Genetic screenings have now also identified heterozygous missense mutations in TREM2 as risk factors for Parkinson's disease (PD), amyotrophic lateral sclerosis (ALS), and fronto-temporal dementia (FTD), in addition to AD (Kleinberger, Sci Transl Med. 2014 Jul. 2; 6(243):243ra86). Thus, functional TREM-2 is required to protect against ageing-related neuroinflammatory and neurodegenerative diseases that cause severe cognitive impairment and dementia.

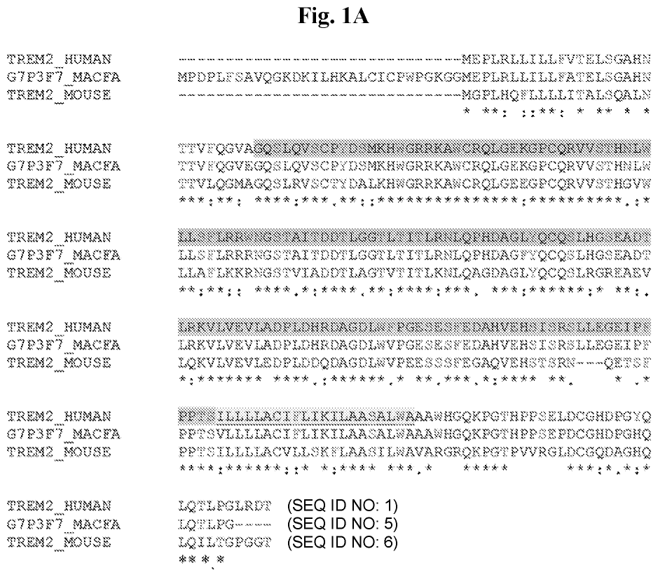

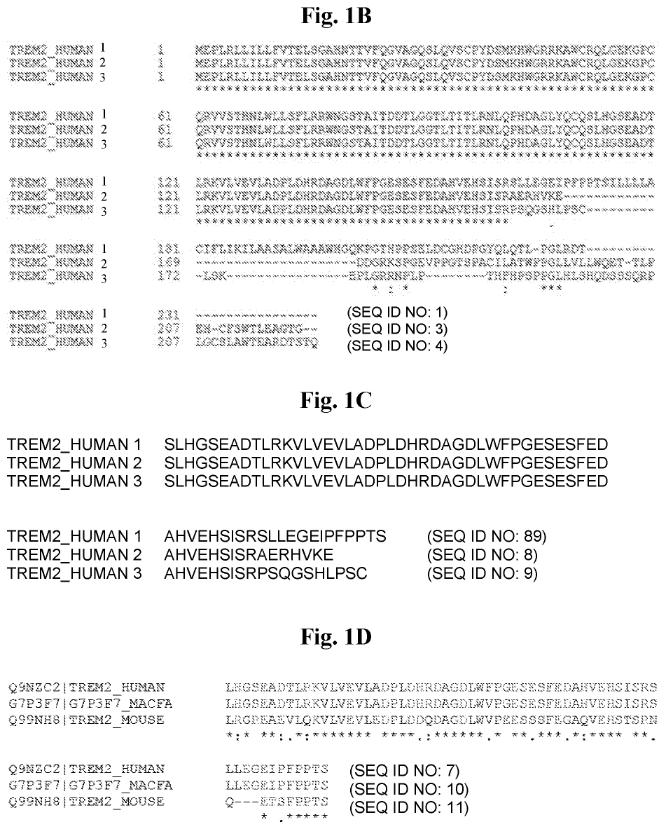

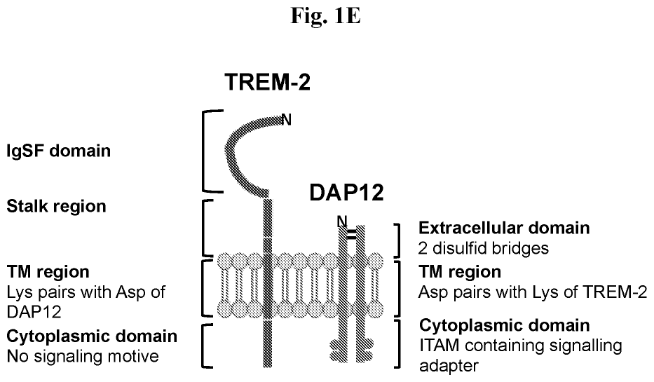

Due to alternative splicing, there are three human TREM2 isoforms, with the isoform 1 being the longest isoform. The amino acid sequences of human TREM2 isoform 1 (SEQ ID NO: 1), human TREM2 isoform 2 (SEQ ID NO: 3), and human TREM2 isoform 3 (SEQ ID NO: 4) were aligned in FIG. 1B. Alignment of the amino acid sequences of the stalk regions of human TREM2 isoform 1 (SEQ ID NO: 89), isoform 2 (SEQ ID NO: 8), and isoform 3 (SEQ ID NO: 9) revealed that the stalk region of human TREM2 isoform 1 shares about 79% sequence identity to the stalk region of human TREM2 isoforms 2 or 3 (FIG. 1C).

The amino acid sequences of human TREM2 isoform 1 (SEQ ID NO: 1), Cyno TREM2 isoform 1 (SEQ ID NO: 5), and mouse TREM2 isoform 1 (SEQ ID NO: 6) were aligned in FIG.1A. Alignment of the amino acid sequences of the stalk regions of human TREM2 isoform 1 (SEQ ID NO: 7), Cyno TREM2 isoform 1 (SEQ ID NO: 10), and mouse TREM2 isoform 1 (SEQ ID NO: 11) revealed that human TREM2 isoform 1 stalk region shares 98% sequence identity to Cyno TREM2 isoform 1 stalk region, and 69% sequence identity to mouse TREM2 isoform 1 stalk region (FIG. 1D). FIG. 1E illustrates the structure of TREM2 and its interaction with the signaling adaptor protein DAP12.

### Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

As used herein, “TREM2” (also known as “triggering receptor expressed on myeloid cells 2”, TREM-2, TREM2a, TREM2b, or TREM2c) refers to a glycoprotein encoded by the TREM2 gene. Human TREM2 belongs to the immunoglobulin superfamily (IgSF), and includes a signal peptide, a single V-type immunoglobulin domain (IgV), a stalk region, a transmembrane domain, and a cytoplasmic tail. The human TREM2 gene is mapped to chromosomal location 6p21.1, and the genomic sequence of human TREM2 gene can be found in GenBank at NC_000006.12. Due to alternative splicing, there are at least three human TREM2 isoforms. The term “human TREM2” is used to refer to any isoform of human TREM2. The protein and mRNA sequences for the longest human TREM2 isoform (isoform 1) are:

Triggering receptor expressed on myeloid cells 2 precursor isoform 1 precursor
[Homo sapiens] (NP_061838.1)
(SEQ ID NO: 1)
MEPLRLLILLFVTELSGAHNTTVFQGVAGQSLQVSCPYDSMKHWGRRKAWC
RQLGEKGPCQRVVSTHNLWLLSFLRRWNGSTAITDDTLGGTLTITLRNLQPHDAGLY
QCQSLHGSEADTLRKVLVEVLADPLDHRDAGDLWFPGESESFEDAHVEHSISRSLLE
GEIPFPPTSILLLLACIFLIKILAASALWAAAWHGQKPGTHPPSELDCGHDPGYQLQTL
PGLRDT
Homo sapiens triggering receptor expressed on myeloid cells 2 (TREM2), transcript
variant 1, mRNA (NM_018965.3)
(SEQ ID NO: 2)
gggcagcgcc tgacatgcct gatcctctct tttctgcagt tcaagggaaa gacgagatct tgcacaaggc
actctgcttc tgcccttggc tggggaaggg tggcatggag cctctccggc tgctcatctt actctttgtc acagagctgt
ccggagccca caacaccaca gtgttccagg gcgtggcggg ccagtccctg caggtgtctt gcccctatga ctccatgaag
cactggggga ggcgcaaggc ctggtgccgc cagctgggag agaagggccc atgccagcgt gtggtcagca cgcacaactt
gtggctgctg tccttcctga ggaggtggaa tgggagcaca gccatcacag acgataccct gggtggcact ctcaccatta
cgctgcggaa tctacaaccc catgatgcgg gtctctacca gtgccagagc ctccatggca gtgaggctga caccctcagg
aaggtcctgg tggaggtgct ggcagacccc ctggatcacc gggatgctgg agatctctgg ttccccgggg agtctgagag
cttcgaggat gcccatgtgg agcacagcat ctccaggagc ctcttggaag gagaaatccc cttcccaccc acttccatcc
ttctcctcct ggcctgcatc tttctcatca agattctagc agccagcgcc ctctgggctg cagcctggca tggacagaag
ccagggacac atccacccag tgaactggac tgtggccatg acccagggta tcagctccaa actctgccag ggctgagaga
cacgtgaagg aagatgatgg gaggaaaagc ccaggagaag tcccaccagg gaccagccca gcctgcatac ttgccacttg
gccaccagga ctccttgttc tgctctggca agagactact ctgcctgaac actgcttctc ctggaccctg gaagcaggga
ctggttgagg gagtggggag gtggtaagaa cacctgacaa cttctgaata ttggacattt taaacactta caaataaatc
caagactgtc atatttagct ggataaaaaa aaaaaaaaaa aaaaaa

The amino acid sequences of human TREM2 isoform 2 (SEQ ID NO: 3) and isoform 3 (SEQ ID NO: 4) are shown in FIG. 1B. In some embodiments, TREM2 protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any of SEQ ID No: 1, 3, or 4, wherein such proteins still have the ligand binding, intracellular singaling, facilitating phagocytosis and degradation of phagocytic material, and other regulatory function of TREM2. The sequences of murine, cyno, and other animal TREM2 proteins are known in the art (for example, NP_112544.1 and NP_001259007.1 for murine TREM2 protein).

The term “extracellular domain” refers to the portion of a transmembrane protein that is exposed on the extracellular side of a lipid bilayer of a cell. Methods for determining the ectodomain of a protein are known in the art (Singer (1990); High et al. (1993), and McVector software, Oxford Molecular). For example, the extracellular domain of human TREM2 protein can include the amino acid residues 14 to 174 of SEQ ID NO: 1 (isoform 1), the amino acid residues 14 to 168 of SEQ ID NO: 3 (isoform 2), or the amino acid residues 14 to 171 of SEQ ID NO: 4 (isoform 3).

The term “ectodomain” of TREM2 refers to a portion of the extracellular domain of TREM2 that is released after sheddase cleavage.

The term “stalk region” of TREM2 refers to a portion of the extracellular domain of TREM2 that connects the V-type immunoglobulin (IgV) domain and the transmembrane domain.

The term “transmembrane domain” refers to the portion of a transmembrane protein that spans the lipid bilayer of a cell. Methods for determining the transmembrane domain of a protein are known in the art (Elofsson et al. (2007) Annu. Rev. Biochem. 76:125-140; Bernsel et al. (2005) Protein Science 14:1723-1728).

The terms “cytoplasmic domain” and “cytoplasmic tail” are used interchangeably and refer to the portion of a transmembrane protein that is on the cytoplasmic side of the lipid bilayer of a cell. Methods for determining the cytoplasmic tail of a protein are known in the art (Elofsson et al. (2007) and Bernsel et al. (2005)).

The terms “cleavage resistant TREM2 mutant” and “TREM2 mutant resistant to sheddase cleavage” are used interchangeablely herein, and refer to a TREM2 mutant that harbors one or more mutations near the cleavage site of a sheddase that cleaves wild type TREM2 (e.g., ADAM17 or ADAM10) and thus have reduced cleavage by the sheddase, e.g., about 10%, 20%, 30%, 40%, 50%, 60%, 70%, 80% or 90% less cleavage by the sheddase, compared to the wild type TREM2 protein under the same condition. A “cleavage resistant TREM2 mutant” can have reduced ectodomain shedding, e.g., about 10%, 20%, 30%, 40%, 50%, 60%, 70%, 80% or 90% less shedding than the wild type TREM2 protein under the same condition. Such cleavage resistant TREM2 mutants can have reduced shedding while retain key TREM2 functions, for example, can still have the ligand binding, intracellular singaling, facilitating phagocytosis and degradation of phagocytic material, and other regulatory functions of TREM2.

As used herein, “DAP12” (also known as TYROBP; KARAP; PLOSL) refers to a transmembrane signaling polypeptide which contains an immunoreceptor tyrosine-based activation motif (ITAM) in its cytoplasmic domain. The protein and mRNA sequences for the longest human DAP12 isoform (isoform 1) are:

TYRO protein tyrosine kinase-binding protein isoform 1 precursor [Homo sapiens]
(NP_003323.1)
(SEQ ID NO: 49)
MGGLEPCSRLLLLPLLLAVSGLRPVQAQAQSDCSCSTVSPGVLAGIVMGDLVLTVLI
ALAVYFLGRLVPRGRGAAEAATRKQRITETESPYQELQGQRSDVYSDLNTQRPYYK
Homo sapiens TYRO protein tyrosine kinase binding protein (TYROBP), transcript
variant 1, mRNA (NM_003332.3)
(SEQ ID NO: 50)
agacttcctc cttcacttgc ctggacgctg cgccacatcc caccggccct tacactgtgg tgtccagcag catccggctt
catgggggga cttgaaccct gcagcaggct cctgctcctg cctctcctgc tggctgtaag tggtctccgt cctgtccagg
cccaggccca gagcgattgc agttgctcta cggtgagccc gggcgtgctg gcagggatcg tgatgggaga cctggtgctg
acagtgctca ttgccctggc cgtgtacttc ctgggccggc tggtccctcg ggggcgaggg gctgcggagg cagcgacccg
gaaacagcgt atcactgaga ccgagtcgcc ttatcaggag ctccagggtc agaggtcgga tgtctacagc gacctcaaca
cacagaggcc gtattacaaa tgagcccgaa tcatgacagt cagcaacatg atacctggat ccagccattc ctgaagccca
ccctgcacct cattccaact cctaccgcga tacagaccca cagagtgcca tccctgagag accagaccgc tccccaatac
tctcctaaaa taaacatgaa gcacaaaaac aaaaaaaaaa aaaaaaaa

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An isolated antibody is substantially free of other antibodies having different antigenic specificities (e.g., an isolated antibody that specifically binds TREM2 is substantially free of antibodies that specifically bind antigens other than TREM2). An isolated antibody that specifically binds a target molecule may, however, have cross-reactivity to the same antigens from other species, e.g., an isolated antibody that specifically binds TREM2 may bind TREM2 molecules from other species. Moreover, an isolated antibody may be substantially free of other cellular material and/or chemicals.

Unless otherwise defined, all technical and scientific terms used herein have the same meaning as commonly understood by one of ordinary skill in the art to which this disclosure pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### TREM2 Mutants Resistant to Sheddase Cleavage

TREM2 undergoes sequential proteolytic processing by ectodomain shedding and intramembrane proteolysis (Wunderlich, J. Biol. Chem. 288, 33027-33036, 2013). During ectodomain shedding, the ectodomain of TREM2 is released by proteases such as members of the ADAM (a disintegrin and metalloproteinase domaincontaining protein) or BACE (b-site APP cleaving enzyme) family (Kleinberger, Sci Transl Med. 2014 Jul. 2; 6(243):243ra86). Soluble fragments of TREM2 (sTREM2) produced by ectodomain shedding have been observed in supernatants of dendritic cell cultures as well as in plasma and CSF samples from patients with noninflammatory neurological diseases and multiple sclerosis (Kleinberger, 2014). Detailed analysis during the course of Alzheimer's disease (AD) revealed that sTREM2 increases early in AD before clinical symptoms appear, peaks in MCI-AD, and stays elevated but at lower levels compared to the MCI-AD stage in AD dementia (Suarez-Calvet, EMBO molecular medicine 8, 466-476, 2016).

The data presented here identified ADAM17 as the main sheddase responsible for constitutive shedding (Example 2). ADAM17 ablation reduced TREM2 constitutive shedding and increased cell surface TREM2 (Example 3). After phorbol-myristate-acid (PMA) treatment, additional shedding mechanisms come into play, one of which might involve ADAM10 (Example 3). Two areas that are important for PMA induced shedding of TREM2 were identified: a membrane proximal at amino acids 169-172 and a membrane distal in the region amino acids 156-164 (Example 4). HPLC analysis showed that the H157-S158 bond in TREM2 is the main cleavage site for ADAM17 (Example 5). TREM2 ectodomain shed from cells was characterized and confirmed the main cleavage site between H157 and S158 (Example 6). No O-glycosylation was identified at the positions close to the cleavage site: S160 or S168 (Example 7). TREM2 mutants with mutations at the sheddase cleavage site show increased cell surface expression and resistance to sheddase cleavage (Example 8).

Provided herein are TREM2 mutants resistant to sheddase cleavage (or “cleavage resistant TREM2 mutants”), e.g., human TREM2 mutants resistant to sheddase cleavage (or “cleavage resistant human TREM2 mutants”). In some embodiments, the cleavage resistant TREM2 mutants are resistant to ADAM17 or ADAM 10 cleavage. In some embodiments, the cleavage resistant TREM2 mutants comprise a mutant stalk region. For example, the cleavage resistant TREM2 mutants can harbor one or more mutations near the sheddase cleavage site. In some embodiments, the cleavage resistant TREM2 mutants comprise one or more mutations near the ADAM17 cleavage site between H157 and S158.

In some embodiments, a human TREM2 mutant resistant to sheddase cleavage comprises a stalk region comprising any one of the amino acid sequences provided in Table 1. In some embodiments, a human TREM2 mutant resistant to sheddase cleavage comprises a stalk region comprising an amino acid sequence selected from any one of SEQ ID NOs: 33-40. In some embodiments, a human TREM2 mutant resistant to sheddase cleavage comprises a stalk region consisting of an amino acid sequence selected from any one of SEQ ID NOs: 33-40. In some embodiments, a human TREM2 mutant resistant to sheddase cleavage comprises a stalk region comprising an amino acid sequence selected from any one of SEQ ID NOs: 33-35. In some embodiments, a human TREM2 mutant resistant to sheddase cleavage comprises a stalk region consisting of an amino acid sequence selected from any one of SEQ ID NOs: 33-35. In some embodiments, a human TREM2 mutant resistant to sheddase cleavage comprises a stalk region comprising an amino acid sequence selected from any one of SEQ ID NOs: 33, 36, 40. In some embodiments, a human TREM2 mutant resistant to sheddase cleavage comprises a stalk region consisting of an amino acid sequence selected from any one of SEQ ID NOs: 33, 36, 40. In some embodiments, a human TREM2 mutant resistant to sheddase cleavage comprises a stalk region comprising SEQ ID NO: 33. In some embodiments, a human TREM2 mutant resistant to sheddase cleavage comprises a stalk region consisting of SEQ ID NO: 33.

TABLE 1
Exemplary amino acid sequences of the stalk region of human TREM2 mutant
resistant to sheddase cleavage

| | SEQ | |
|---|---|---|
| | ID | |
| Name | NO | Sequence |
| T2-IPD | 33 | SLHGSEADTLRKVLVEVLADPLDHRDAGDLWFPGESESFEDAHVE |
| | | IPDSRSLLEGEIPFPPTS |
| T2-IPP | 34 | SLHGSEADTLRKVLVEVLADPLDHRDAGDLWFPGESESFEDAHVE |
| | | IPPSRSLLEGEIPFPPTS |
| T2-IDP | 35 | SLHGSEADTLRKVLVEVLADPLDHRDAGDLWFPGESESFEDAHVE |
| | | IDPSRSLLEGEIPFPPTS |
| TRUNC3 (159- | 36 | SLHGSEADTLRKVLVEVLADPLDHRDAGDLWFPGESESFEDAHVE |
| 174 deletion) | | HS |
| T2del 11-16 | 37 | SLHGSEADTLRKVLVEVLADPLDHRDAGDLWFPGESESFEDAHVE |
| | | HSEGEIPFPPTS |
| T2-YGG | 38 | SLHGSEADTLRKVLVEVLADPLDHRDAGDLWFPGESESFEDAHVY |
| | | GGWGGWGPEGEIPFPPTS |
| T2-WFR | 39 | SLHGSEADTLRKVLVEVLADPLDHRDAGDLWFPGESESFEDAHVE |
| | | HSISRSLLEGEIWFRWTS |
| T2-double | 40 | SLHGSEADTLRKVLVEVLADPLDHRDAGDLWFPGESESFEDAHVY |
| | | GGWGGWGPEGEIWFRWTS |

In some embodiments, a human TREM2 mutant resistant to sheddase cleavage comprises an amino acid sequence selected from the amino acid sequences provided in Table 2. In some embodiments, a human TREM2 mutant resistant to sheddase cleavage comprises an amino acid sequence selected from any one of SEQ ID NOs: 41-48. In some embodiments, a human TREM2 mutant resistant to sheddase cleavage consists of an amino acid sequence selected from any one of SEQ ID NOs: 41-48. In some embodiments, a human TREM2 mutant resistant to sheddase cleavage comprises an amino acid sequence selected from any one of SEQ ID NOs: 41-43. In some embodiments, a human TREM2 mutant resistant to sheddase cleavage consists of an amino acid sequence selected from any one of SEQ ID NOs: 41-43. In some embodiments, a human TREM2 mutant resistant to sheddase cleavage comprises an amino acid sequence selected from any one of SEQ ID NOs: 41, 44, 48. In some embodiments, a human TREM2 mutant resistant to sheddase cleavage consists of an amino acid sequence selected from any one of SEQ ID NOs: 41, 44, 48. In some embodiments, a human TREM2 mutant resistant to sheddase cleavage comprises SEQ ID NO: 41. In some embodiments, a human TREM2 mutant resistant to sheddase cleavage consists of SEQ ID NO: 41.

Also provided herein are polypeptides comprising an amino acid sequence selected from any one of SEQ ID NOs: 33-40. In some embodiments, the polypepetide comprising an amino acid sequence selected from any one of SEQ ID NOs: 33-40. In some embodiments, the polypepetide comprising an amino acid sequence selected from any one of SEQ ID NOs: 41-48. In some embodiments, the polypepetide consisting of an amino acid sequence selected from any one of SEQ ID NOs: 41-48. In some embodiments, the polypepetide comprising an amino acid sequence selected from any one of SEQ ID NOs: 41-43. In some embodiments, the polypepetide consisting of an amino acid sequence selected from any one of SEQ ID NOs: 41-43.

TABLE 2
Exemplary amino acid sequences of human TREM2 mutant resistant to
sheddase cleavage

| | SEQ | |
|---|---|---|
| | ID | |
| Name | NO | Sequence |
| T2-IPD | 41 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEIPDSRSLLEGEIPFPPTSILLLLACI |
| | | FLIKILAASALWAAAWHGQKPGTHPPSELDCGHDPGYQLQTLPGL |
| | | RDT |
| T2-IPP | 42 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEIPPSRSLLEGEIPFPPTSILLLLACIF |
| | | LIKILAASALWAAAWHGQKPGTHPPSELDCGHDPGYQLQTLPGLR |
| | | DT |
| T2-IDP | 43 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEIDPSRSLLEGEIPFPPTSILLLLACI |
| | | FLIKILAASALWAAAWHGQKPGTHPPSELDCGHDPGYQLQTLPGL |
| | | RDT |
| TRUNC3 (159- | 44 | MEPLRLLILLFVTELSGAHNTTVFQGVAGQSLQVSCPYDSMKHWG |
| 174 deletion) | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEHSILLLLACIFLIKILAASALWAAA |
| | | WHGQKPGTHPPSELDCGHDPGYQLQTLPGLRDT |
| T2del 11-16 | 45 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEHSEGEIPFPPTSILLLLACIFLIKILA |
| | | ASALWAAAWHGQKPGTHPPSELDCGHDPGYQLQTLPGLRDT |
| T2-YGG | 46 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVYGGWGGWGPEGEIPFPPTSILLLL |
| | | ACIFLIKILAASALWAAAWHGQKPGTHPPSELDCGHDPGYQLQTLP |
| | | GLRDT |
| T2-WFR | 47 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVEHSISRSLLEGEIWFRWTSILLLLA |
| | | CIFLIKILAASALWAAAWHGQKPGTHPPSELDCGHDPGYQLQTLPG |
| | | LRDT |
| T2-double | 48 | MEPLRLLILLFVTELSGAHNTTVFQGVAGQSLQVSCPYDSMKHWG |
| | | RRKAWCRQLGEKGPCQRVVSTHNLWLLSFLRRWNGSTAITDDTL |
| | | GGTLTITLRNLQPHDAGLYQCQSLHGSEADTLRKVLVEVLADPLD |
| | | HRDAGDLWFPGESESFEDAHVYGGWGGWGPEGEIWFRWTSILL |
| | | LLACIFLIKILAASALWAAAWHGQKPGTHPPSELDCGHDPGYQLQ |
| | | TLPGLRDT |

### Nucleic Acids Encoding Human TREM2 Mutants, Vectors, and Cells

The present disclosure also provides nucleic acids encoding a human TREM2 mutant resistant to sheddase cleavage, vectors for expression of a human TREM2 mutant resistant to sheddase cleavage, and cells containing such expression vectors. In other aspects, the disclosure provides a sequence encoding a human TREM2 mutant resistant to sheddase cleavage, and expression vectors and host cells comprising such a polynucleotide.

In some embodiments, provided herein are nucleic acids comprising a sequence encoding a human TREM2 mutant resistant to sheddase cleavage that comprises a stalk region comprising any one of the amino acid sequences provided in Table 1. In some embodiments, provided herein are nucleic acids comprising a sequence encoding a human TREM2 mutant resistant to sheddase cleavage that comprises a stalk region comprising an amino acid sequence selected from any one of SEQ ID NOs: 33-40. In some embodiments, provided herein are nucleic acids comprising a sequence encoding a human TREM2 mutant resistant to sheddase cleavage that comprises a stalk region consisting of an amino acid sequence selected from any one of SEQ ID NOs: 33-40. In some embodiments, provided herein are nucleic acids comprising a sequence encoding a human TREM2 mutant resistant to sheddase cleavage that comprises a stalk region comprising an amino acid sequence selected from any one of SEQ ID NOs: 33-35. In some embodiments, provided herein are nucleic acids comprising a sequence encoding a human TREM2 mutant resistant to sheddase cleavage that comprises a stalk region consisting of an amino acid sequence selected from any one of SEQ ID NOs: 33-35. In some embodiments, provided herein are nucleic acids comprising a sequence encoding a human TREM2 mutant resistant to sheddase cleavage that comprises a stalk region comprising an amino acid sequence selected from any one of SEQ ID NOs: 33, 36, 40. In some embodiments, provided herein are nucleic acids comprising a sequence encoding a human TREM2 mutant resistant to sheddase cleavage that comprises a stalk region consisting of an amino acid sequence selected from any one of SEQ ID NOs: 33, 36, 40. In some embodiments, provided herein are nucleic acids comprising a sequence encoding a human TREM2 mutant resistant to sheddase cleavage that comprises a stalk region comprising SEQ ID NO: 33. In some embodiments, provided herein are nucleic acids comprising a sequence encoding a human TREM2 mutant resistant to sheddase cleavage that comprises a stalk region consisting of SEQ ID NO: 33.

In some embodiments, provided herein are nucleic acids comprising a sequence encoding a human TREM2 mutant resistant to sheddase cleavage that comprises an amino acid sequence selected from the amino acid sequences provided in Table 2. In some embodiments, provided herein are nucleic acids comprising a sequence encoding a human TREM2 mutant resistant to sheddase cleavage that comprises an amino acid sequence selected from any one of SEQ ID NOs: 41-48. In some embodiments, provided herein are nucleic acids comprising a sequence encoding a human TREM2 mutant resistant to sheddase cleavage that consists of an amino acid sequence selected from any one of SEQ ID NOs: 41-48. In some embodiments, provided herein are nucleic acids comprising a sequence encoding a human TREM2 mutant resistant to sheddase cleavage that comprises an amino acid sequence selected from any one of SEQ ID NOs: 41-43. In some embodiments, provided herein are nucleic acids comprising a sequence encoding a human TREM2 mutant resistant to sheddase cleavage that consists of an amino acid sequence selected from any one of SEQ ID NOs: 41-43. In some embodiments, provided herein are nucleic acids comprising a sequence encoding a human TREM2 mutant resistant to sheddase cleavage that comprises an amino acid sequence selected from any one of SEQ ID NOs: 41, 44, 48. In some embodiments, provided herein are nucleic acids comprising a sequence encoding a human TREM2 mutant resistant to sheddase cleavage that consists of an amino acid sequence selected from any one of SEQ ID NOs: 41, 44, 48. In some embodiments, provided herein are nucleic acids comprising a sequence encoding a human TREM2 mutant resistant to sheddase cleavage that comprises SEQ ID NO: 41. In some embodiments, provided herein are nucleic acids comprising a sequence encoding a human TREM2 mutant resistant to sheddase cleavage that consists of SEQ ID NO: 41.

In some embodiments, provided herein are nucleic acids comprising a sequence encoding a human TREM2 mutant resistant to sheddase cleavage, wherein the sequence comprises any one of the sequences provided in Table 3. In some embodiments, provided herein are nucleic acids comprising a sequence encoding a human TREM2 mutant resistant to sheddase cleavage, wherein the sequence comprises any one of SEQ ID NOs: 67-74. In some embodiments, provided herein are nucleic acids comprising a sequence encoding a human TREM2 mutant resistant to sheddase cleavage, wherein the sequence comprises any one of SEQ ID NOs: 67-69. In some embodiments, provided herein are nucleic acids comprising a sequence encoding a human TREM2 mutant resistant to sheddase cleavage, wherein the sequence comprises any one of SEQ ID NOs: 67, 70, or 74. In some embodiments, provided herein are nucleic acids comprising a sequence encoding a human TREM2 mutant resistant to sheddase cleavage, wherein the sequence comprises SEQ ID NO: 67.

In some embodiments, provided herein are nucleic acids comprising any one of SEQ ID NOs: 67-74. In some embodiments, provided herein are nucleic acids comprising any one of SEQ ID NOs: 67-69. In some embodiments, provided herein are nucleic acids comprising any one of SEQ ID NOs: 67, 70, or 74. In some embodiments, provided herein are nucleic acids comprising SEQ ID NO: 67.

TABLE 3
Exemplary nucleic acid sequences encoding the stalk region of
human TREM2 mutant resistant to sheddase cleavage

| | SEQ | |
|---|---|---|
| Name | ID NO | Sequences |
| T2-IPD | 67 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGGAAATTCCGGATAGCCGCAGCCTGCTGGAAGGC |
| | | GAAATTCCGTTTCCGCCGACCAGC |
| T2-IPP | 68 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGGAAATTCCGCCGAGCCGCAGCCTGCTGGAAGGC |
| | | GAAATTCCGTTTCCGCCGACCAGC |
| T2-IDP | 69 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGGAAATTGATCCGAGCCGCAGCCTGCTGGAAGGC |
| | | GAAATTCCGTTTCCGCCGACCAGC |
| TRUNC3 (159- | 70 | AGCCTGCATGGCAGCGAAGCGGATACCCTGCGCAAAGTGCT |
| 174 deletion) | | GGTGGAAGTGCTGGCGGATCCGCTGGATCATCGCGATGCGG |
| DNA sequence | | GCGATCTGTGGTTTCCGGGCGAAAGCGAAAGCTTTGAAGATG |
| | | CGCATGTGGAACATAGC |
| T2del 11-16 | 71 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGGAACATAGCGAAGGCGAAATTCCGTTTCCGCCGA |
| | | CCAGC |
| T2-YGG | 72 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGTATGGCGGCTGGGGCGGCTGGGGCCCGGAAGGC |
| | | GAAATTCCGTTTCCGCCGACCAGC |
| T2-WFR | 73 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGGAACATAGCATTAGCCGCAGCCTGCTGGAAGGC |
| | | GAAATTTGGTTTCGCTGGACCAGC |
| T2-double | 74 | AGCCTGCATGGCAGCGAAGCGGATACCCTGCGCAAAGTGCT |
| DNA sequence | | GGTGGAAGTGCTGGCGGATCCGCTGGATCATCGCGATGCGG |
| | | GCGATCTGTGGTTTCCGGGCGAAAGCGAAAGCTTTGAAGATG |
| | | CGCATGTGTATGGCGGCTGGGGCGGCTGGGGCCCGGAAGGC |
| | | GAAATTTGGTTTCGCTGGACCAGC |

Provided herein are vectors (e.g., expression vectors) that can be employed to express a human TREM2 mutant resistant to sheddase cleavage. The term “expression vector” refers to a carrier nucleic acid molecule into which a desired coding sequence can be inserted for introduction into a cell where it can be expressed. Expression vector can be a DNA vector, an RNA vector, a plasmid, a cosmid, or a viral vector, or artificial chromosomes (see, e.g., Harrington et al., Nat Genet 15:345, 1997). For example, nonviral vectors useful for expression of a polypeptide in mammalian (e.g., human) cells include pThioHis A, B & C, pcDNA3. 1/His, pEBVHis A, B & C (Invitrogen, San Diego, Calif.), MPSV vectors, and numerous other vectors known in the art for expressing other proteins. In some embodiments, the expression vector can be capable of autonomous replication or it can integrate into a host DNA. In some embodiments, the expression vector further comprises a selectable marker.

Useful viral vectors include, but are not limited to, vectors based on any of the following viruses: adenovirus, adeno-associated virus, Herpes Simplex Virus (HSV), parvovirus, retrovirus, lentivirus, vaccinia virus, Sinbis virus, influenza virus, reovirus, Newcastle disease virus (NDV), measles virus, vesicular stomatitis virus (VSV), poliovirus, poxvirus, Seneca Valley virus, coxsackievirus, enterovirus, myxoma virus, or maraba virus.

In some embodiments, the expression vector is a lentiviral vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In some embodiments, the expression vector is an adeno-associated virus (AAV) vector, e.g., a recombinant AAV (rAAV) vector. “AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes, for example, AAV type 1 (AAV1), AAV type 2 (AAV2), AAV type 3 (AAV3), AAV type 4 (AAV4), AAV type 5 (AAVS), AAV type 6 (AAV6), AAV type 7 (AAV7), AAV type 8 (AAV8), AAV type 9 (AAV9), AAV type 10 (AAV10, including AAVrh10), AAV type 12 (AAV12), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, and so on.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession NOs. NC-002077 (AAV1), AF063497 (AAV1), NC-001401 (AAV2), AF043303 (AAV2), NC-001729 (AAV3), NC-001829 (AAV4), U89790 (AAV4), NC-006152 (AAVS), AF513851 (AAV7), AF513852 (AAV8), and NC-006261 (AAV8); or in publications such as WO2005033321 (AAV1-9), the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al.,(1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; and U.S. Pat. No. 6,156,303.

An “rAAV vector” as used herein refers to an AAV vector comprising a polynucleotide sequence not of AAV origin (i.e., a polynucleotide heterologous to AAV), typically a sequence of interest for the genetic transformation of a cell. In some embodiments, the heterologous polynucleotide may be flanked by at least one, and sometimes by two, AAV inverted terminal repeat (ITR) sequences. The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector.” Thus, production of rAAV particle necessarily includes production of rAAV vector, as such a vector is contained within an rAAV particle.

In some embodiments, the expression vector can be a recombinant DNA molecule containing a nucleic acid encoding a human TREM2 mutant resistant to sheddase cleavage. “Recombinant,” as used herein means that the vector, polynucleotide, polypeptide or cell is the product of various combinations of cloning, restriction or ligation steps (e.g. relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

The recombinant expression vector typically includes one or more regulatory sequences operatively linked to the nucleic acid sequence to be expressed. The term “regulatory sequence” includes promoters, enhancers, and other expression control elements (e.g., polyadenylation signals). Regulatory sequences include those which direct constitutive expression of a nucleotide sequence, as well as tissue-specific regulatory and/or inducible sequences. Expression vectors can also include elements designed to optimize messenger RNA stability and translatability in host cells, and/or drug selection markers for establishing permanent, stable cell clones expressing a human TREM2 mutant. The design of the expression vector can depend on such factors as the choice of the host cell to be transformed, the level of expression of protein desired, and the like. General methods for generating such recombinant expression vectors can be found in Sambrook and Russell eds. (2001) Molecular Cloning: A Laboratory Manual, 3rd edition; the series Ausubel et al. eds. (2007 with updated through 2010) Current Protocols in Molecular Biology, among others known in the art.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

The promoters employed can be constitutive, inducible, synthetic, tissue- or cell-specific, and/or useful under the appropriate conditions to direct high level expression of the introduced DNA segment, such as is advantageous in the large-scale production of recombinant proteins and/or peptides. In addition, other regulatory elements may also be incorporated to improve expression of a nucleic acid encoding a TREM2 mutant protein, e.g., enhancers, ribosomal binding site, transcription termination sequences, and the like.

In some embodiments, a constitutive promoter is employed to provide constant expression of a TREM2 mutant protein. Examples of a constitutive promoter include, but not limited to, the immediate early cytomegalovirus (CMV) promoter, the simian virus 40 (SV40) early promoter, mouse mammary tumor virus (MMTV) promoter, human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter.

Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, a tissue- or cell-specific promoter is employed to provide expression of a TREM2 mutant protein only in specific tissues or cells. The identity of tissue- or cell-specific promoters or elements, as well as assays to characterize their activities, is well known to those of skill in the art. Examples include the human LIMK2 gene (Nomoto et al. 1999, Gene, 236(2):259-271), the somatostatin receptor 2 gene (Kraus et al., 1998, FEES Lett., 428(3): 165-170), murine epididymal retinoic acid-binding gene (Lareyre et al., 1999, J. Biol. Chem., 274(12):8282-8290), human CD4 (Zhao-Emonet et al., 1998, Biochirn. Biophys. Acta, 1442(2-3): 109-119), mouse alpha2 (XI) collagen (Tsumaki, et al., 1998, J. Biol. Chem., 273(36):22861-22864), D1A dopamine receptor gene (Lee, et al., 1997, J. Auton. Nerv. Syst., 74(2-3):86-90), insulin-like growth factor II (Wu et al., 1997, Biochem. Biophys. Res. Commun., 233(1):221-226), human platelet endothelial cell adhesion molecule-1 (Almendro et al., 1996, J. Immunol., 157(12):5411-5421), muscle creatine kinase (MCK) promoter (Wang et al., Gene Ther. 2008 November; 15(22):1489-99).

In some embodiments, the promoter is a cell-type specific promoter. For example, a promoter can be employed to drive TREM2 expression specifically in a macrophage, a dendritic cell, or a microglia. In some embodiments, a specific promoter is employed to provide TREM2 protein expression in a microglia. The promoters that can direct TREM2 protein expression in microglia include but not limited to a TREM2 promoter, TMEM119 promoter, Hexb promoter, IBA1 promoter, CD45 promoter, CD11b promoter, Cst7 promoter, Lpl promoter, Csf1 promoter, Cs1R promoter, Itgax promoter, Clec7a promoter, Lilrb4 promoter, Tyrobp promoter, Ctsb promoter, Ctsd promoter, B2m promoter, Lyz2 promoter, Cx3cr1 promoter, Cst3 promoter, Ctss promoter, P2ryl2 promoter, C1qa promoter, C1qb promoter, Axl promoter, Timp2 promoter, Ctsl promoter, Gnas promoter, Cd9 promoter, Fth1 promoter, Tmsb4x promoter. In some embodiments, the expression vector includes a promoter selected from a TREM2 promoter, TMEM119 promoter, Hexb promoter, IBA1 promoter, CD45 promoter, CD11b promoter, Cst7 promoter, Lpl promoter, Csf1 promoter, Cs1R promoter, Itgax promoter, Clec7a promoter, Lilrb4 promoter, Tyrobp promoter, Ctsb promoter, Ctsd promoter, B2m promoter, Lyz2 promoter, Cx3cr1 promoter, Cst3 promoter, Ctss promoter, P2ryl2 promoter, C1qa promoter, or C1qb promoter. In some embodiments, the expression vector includes a TREM2 promoter.

In some embodiments, a synthetic promoter is employed to provide expression of a TREM2 mutant protein. Synthetic promoters can greatly exceed the transcriptional potencies of natural promoters. For example, the synthetic promoters that do not get shut off or reduced in activity by the endogenous cellular machinery or factors can be selected. Other elements, including trans-acting factor binding sites and enhancers may be inserted into the synthetic promoter to improve transcriptional efficiency. Synthetic promoters can be rationally designed and chemically synthesized to combine the best features of both synthetic and biological promoters. Synthetic oligos are annealed and ligated through several processes to generate the full-length chemically synthesized promoter. Synthetic promoters can be inducible or cell-type specific promoters. For example, synthetic promoter that can drive TREM2 expression specifically in a macrophage, a dendritic cell, or a microglia can be rationally designed and chemically synthesized.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Expression can employ any appropriate host cells known in the art, for example, mammalian host cells, bacterial host cells, yeast host cells, insect host cells, etc. Both prokaryotic and eukaryotic expression systems are widely available. In some embodiments, the expression system is a mammalian cell expression, such as a CHO cell expression system. In some embodiments, a nucleic acid may be codon-optimized to facilitate expression in a desired host cell. It will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, Proc. Natl. Acad. Sci. USA, 94(8):3596-601).

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences. Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

To propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In some embodiments, provided herein are expression vectors comprising a sequence encoding a human TREM2 mutant resistant to sheddase cleavage, e.g., ADAM17 or ADAM10 cleavage. In some embodiments, provided herein are expression vectors comprising a sequence encoding a human TREM2 mutant that comprises a stalk region comprising an amino acid sequence selected from any one of SEQ ID NOs: 33-40. In some embodiments, provided herein are expression vectors comprising a sequence encoding a human TREM2 mutant that comprises a stalk region consisting of an amino acid sequence selected from any one of SEQ ID NOs: 33-40. In some embodiments, provided herein are expression vectors comprising a sequence encoding a human TREM2 mutant that comprises a stalk region comprising an amino acid sequence selected from any one of SEQ ID NOs: 33-35. In some embodiments, provided herein are expression vectors comprising a sequence encoding a human TREM2 mutant that comprises a stalk region consisting of an amino acid sequence selected from any one of SEQ ID NOs: 33-35. In some embodiments, provided herein are expression vectors comprising a sequence encoding a human TREM2 mutant that comprises an amino acid sequence selected from any one of SEQ ID NOs: 41-48. In some embodiments, provided herein are expression vectors comprising a sequence encoding a human TREM2 mutant that consists of an amino acid sequence selected from any one of SEQ ID NOs: 41-48.

In some embodiments, the expression vector comprises a promoter that provides TREM2 protein expression in a macrophage, a dendritic cell, or a microglia. In some embodiments, the expression vector comprises a promoter selected from a TREM2 promoter, TMEM119 promoter, Hexb promoter, IBA1 promoter, CD45 promoter, CD11b promoter, Cst7 promoter, Lpl promoter, Csf1 promoter, Cs1R promoter, Itgax promoter, Clec7a promoter, Lilrb4 promoter, Tyrobp promoter, Ctsb promoter, Ctsd promoter, B2m promoter, Lyz2 promoter, Cx3cr1 promoter, Cst3 promoter, Ctss promoter, P2ryl2 promoter, C1qa promoter, or C1qb promoter. In some embodiments, the expression vector comprises a TREM2 promoter.

In some embodiments, the expression vector comprises a polyadenylation signal. In some embodiments, the expression vector comprises a selectable marker.

In some embodiments, the expression vector further comprises a second sequence encoding DAP12 protein. In some embodiments, the DAP12 protein comprises SEQ ID NO: 49. In some embodiments, the DAP12 protein consists of SEQ ID NO: 49.

In some embodiments, the expression vectors for expressing both a TREM2 polypeptide and a DAP12 polypeptide can comprise an internal ribosome entry site (IRES) upstream of the DAP12-coding sequence. IRES elements are able to bypass the ribosome scanning model of 5′-methylated Cap dependent translation and begin translation at internal sites (Pelletier and Sonenberg, 1988, Nature, 334:320-325). IRES elements from two members of the picornavirus family (polio and encephalomyocarditis) have been described (Pelletier and Sonenberg, 1988), as well an IRES from a mammalian message (Macejak and Sarnow, 1991, Nature, 353:90-94,1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

In some embodiments, the expression vectors for expressing both a TREM2 polypeptide and a DAP12 polypeptide comprise a 2A sequence upstream of the DAP12-coding sequence. The 2A oligopeptide sequence was first characterized from the positive-stranded RNA picornavirus Foot-and-Mouth Disease Virus (FMDV); and FMDV 2A or F2A was shown to mediate a co-translational ‘cleavage’ between the upstream (capsid proteins) and downstream (RNA replication proteins) domains of the FMDV polyprotein (Ryan M D, EMBO J 1994; 134: 928-933; Ryan M D, J Gen Virol 1991; 72: 2727-2732; Donnelly M L L, J Gen Virol 1997; 78:13-21; Donnelly M L L, J Gen Virol 2001; 82:1013-1025). Active 2A sequences were characterized in the genomes of viruses from other genera of the picornaviruses, plus ‘2A-like’ sequences found within the genomes of a range of different RNA viruses and non-LTR retrotransposons (Donnelly M L L, J Gen Virol 2001; 82:1027-1041; Heras S R, Cell Mol Life Sci 2006; 63:1449-1460; Luke G A, J Gen Virol 2008; 89:1036-1042; Odon V, Mol Biol Evol 2013; 30:1955-1965; Luke G A, Mob Gen Elements 2014; 3:e27525). These 2A or “2A-like” oligopeptide sequences were shown to mediate a translational ‘recoding’ event referred-to as ‘ribosome skipping’, ‘stop carry-on’ or ‘stop-go’ translation (Atkins J F, RNA 2007; 13:1-8).

As used herein, a “2A sequence” refers to any nucleic acid sequence encoding a 2A or “2A-like” oligopeptide serving as a linker between two proteins, allowing autonomous intraribosomal self-processing of polyproteins (See e.g., de Felipe. Genetic Vaccines and Ther. 2:13 (2004); deFelipe et al. Traffic 5:616-626 (2004)). These oligopeptides allow co-expression of multiple proteins from a single vector. Many 2A elements are known in the art. For example, viral 2A sequences have been described in U.S. Pat. Nos. 9,175,311, 8,865,881, 7,939,059, 7,947,493, all of which are incorporated by reference herein. For example, a viral 2A sequence can be a picornaviral, a tetraviral 2A sequence, or a combination thereof. A picornaviral 2A sequence can be selected from any one of the Enteroviral 2A sequences, Rhinoviral 2A sequences, Cardioviral 2A sequences, Aphthoviral 2A sequences, Hepatoviral 2A sequences, Erboviral 2A sequences, Kobuviral 2A sequences, Teschoviral 2A sequences, and the Parechoviral 2A sequences. A tetraviral 2A sequences can be selected from any of the Betatetraviral 2A sequences or Omegatetraviral 2A sequences. Examples of 2A sequences that can be used in the methods and system disclosed herein, without limitation, include 2A sequences from the foot-and-mouth disease virus (F2A), equine rhinitis A virus (E2A), Thosea asigna virus (T2A), and porcine teschovirus-1 (P2A). In some embodiments, a 2A sequence encodes a viral 2A oligopeptide selected from T2A (EGRGSLLTCGDVEENPGP (SEQ ID NO: 52) or GSGEGRGSLLTCGDVEENPGP (SEQ ID NO: 53)), P2A (ATNFSLLKQAGDVEENPGP (SEQ ID NO: 54) or GSGATNFSLLKQAGDVEENPGP (SEQ ID NO: 55)), E2A (QCTNYALLKLAGDVESNPGP (SEQ ID NO: 56) or GSGQCTNYALLKLAGDVESNPGP (SEQ ID NO: 57)), or F2A (VKQTLNFDLLKLAGDVESNPGP (SEQ ID NO: 58) or GSGVKQTLNFDLLKLAGDVESNPGP (SEQ ID NO: 59)).

Non-viral 2A sequence has been described in U.S. Pat. No. 8,945,876, which is incorporated by reference herein. For example, a non-viral 2A sequence can be a sea urchin (Strongylocentrotus purpuratus) 2A sequence (DGFCILYLLLILLMRSGDVETNPGP) (SEQ ID NO: 60); a sponge (Amphimedon queenslandica) 2A sequence (LLCFMLLLLLSGDVELNPGP (SEQ ID NO: 61) or HHFMFLLLLLAGDIELNPGP (SEQ ID NO: 62)); an acorn worm (Saccoglossus kowalevskii) 2A sequence (WFLVLLSFILSGDIEVNPGP (SEQ ID NO: 63)); or an amphioxus (Branchiostoma floridae) 2A sequence (KNCAMYMLLLSGDVETNPGP (SEQ ID NO: 64) or MVISQLMLKLAGDVEENPGP (SEQ ID NO: 65)). In some embodiments, the 2A sequence is a naturally occurring or synthetic sequence that includes the 2A consensus sequence D-X-E-X-NPGP (SEQ ID NO: 66), in which X is any amino acid residue.

In some embodiments, the expression vector comprises a 2A sequence encoding a 2A oligopeptide selected from any one of SEQ ID NOs: 52-66.

In some embodiments, a nucleic acid encoding a human TREM2 mutant may also include a sequence encoding a secretion signal sequence so that the polypeptide is secreted from the host cell. Such a sequence can be provided by the vector, or as part of the TREM2 nucleic acid that is present in the vector.

Generation of an expression vector can utilize a vector that includes a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22, agent-enhanced uptake of DNA, and ex vivo transduction. For long-term, high- yield production of recombinant proteins, stable expression will often be desired. For example, cell lines which stably express polypeptides can be prepared using expression vectors which contain viral origins of replication or endogenous expression elements and a selectable marker gene.

Also provided herein are cells that include any of the expression vectors described herein. In some embodiments, such cells comprising an expression vector for expressing a human TREM2 mutant resistant to sheddase cleavage. In some embodiments, such cells comprising an expression vector for expressing both a human TREM2 mutant and a human DAP12 protein. In some embodiments, such cells comprising an expression vector for expressing a human TREM2 mutant and a second expression vector for expressing a human DAP12 protein. Such cells can be a host cell or a therapeutic cell.

In some embodiments, the disclosure features a host cell that includes a nucleic acid molecule described herein. A host cell can be used to produce or express a human TREM2 mutant resistant to sheddase cleavage described herein. The terms “host cell” and “recombinant host cell” are used interchangeably herein, which refer to not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a protein can be expressed in bacterial cells (such as E. coli), insect cells, yeast cells, or mammalian cells (such as Chinese hamster ovary cells (CHO) or COS cells e.g., COS-7 cells, CV-1 origin SV40 cells; Gluzman (1981) Cell23:175-182). Other suitable host cells are known to those skilled in the art.

A host cell can be used to produce or express a human TREM2 mutant described herein. Accordingly, the disclosure also features methods for producing a human TREM2 mutant using a host cell. In one embodiment, the method includes culturing the host cell (into which a recombinant expression vector encoding a protein has been introduced) in a suitable medium, such that a human TREM2 mutant is produced. In another embodiment, the method further includes isolating a human TREM2 mutant from the medium or the host cell.

In some embodiments, the disclosure features a therapeutic cell that includes a nucleic acid molecule described herein. As used herein, the term “therapeutic cell” refers to a cell that has been genetically engineered to express a human TREM2 mutant resistant to sheddase cleavage. Such a therapeutic cell can be a human cell, e.g., a macrophage, a dendritic cell, or a microglia.

In some embodiments, such a therapeutic cell expresses a detectable marker, e.g., a fluorescent molecule (e.g., fluorescein, Texas red, rhodamine, green fluorescent protein, and the like), an enzyme (e.g., horse radish peroxidase, alkaline phosphatase), a luminescent molecule (e.g., luciferase), a radioactive molecule (e.g., ³H, ¹²⁵I, ³⁵S, ¹⁴C, or ³²P), or calorimetric labels such as colloidal gold or colored beads. Cells expressing a detectable marker can be traced or visualized by appropriate detection methods such as microscopy, autoradiography, and/or other imaging methods known in the art.

### Methods of Treatment and Therapeutic Use

Provided herein are methods of increasing TREM2 expression in a subject (e.g., a human) by administering to the subject a nucleic acid encoding a human TREM2 mutant resistant to sheddase cleavage as disclosed herein, or a vector or a cell comprising such a nucleic acid. The subject can have a TREM2-related disease or disorder. Since absent cell surface human TREM2 or excessive shedding of TREM2 was associated with human neuroinflammatory and neurodegenerative pathologies, increasing expression of a cleavage resistant human TREM2 mutant using the methods described herein can be used to treat or prevent such a neuroinflammatory or neurodegenerative disease. The nucleic acids encoding a human TREM2 mutant resistant to sheddase cleavage, or vectors or cells comprising such nucleic acids are also suitable for treating or preventing autoimmune, inflammatory, or malignant disorders mediated by or associated with extensive proteolytic cleavage of TREM2.

Nucleic acids or vectors that can increase TREM2 expression level can be identified by screening candidate nucleic acids or vectors using in vitro cell assays, cell free assays, and/or in vivo animal models. For example, cells can be transfected or infected with a candidate nucleic acid or vector. The level of TREM2, which can be the level of a TREM2 polypeptide having the amino acid sequence set forth in SEQ ID NO:1 or a fragment thereof, on the cell surface, can be monitored to determine whether TREM2 level is increased compared with the level of TREM2 polypeptide in untreated cells or cells treated with a control nucleic acid or vector. The level of cell surface human TREM2 in the sample can be determined by an assay known in the art, e.g., by flow cytometry, immunohistochemistry, Western blotting, immunofluorescent assay, radioimmunoassay (RIA), enzyme-linked immunosorbent assay (ELISA), homogeneous time resolved fluorescence (HTRF), or positron emission tomography (PET), or any other immune detection with an antibody or antibody fragment against human TREM2 protein.

Nucleic acids, vectors or cells that can increase the TREM expression level can also be identified by screening candidate nucleic acids, vectors, or cells in non-human mammals (e.g., TREM2 transgenic or TREM2 knockout non-human mammals). For example, TREM2 expression level can be assessed in a group of non-human mammals administered with the nucleic acids, vectors, or cells, and compared with untreated control mammals to determine whether or not administration of the nucleic acids, vectors, or cells results in an increase in the TREM2 level. Non-human mammals include, for example, rodents such as rats, guinea pigs, and mice, and farm animals such as pigs, sheep, goats, horses, and cattle. Non-human mammals also can be designed to lack endogenous nucleic acid encoding a TREM2 polypeptide or to contain truncated or disrupted endogenous TREM2 nucleic acid (e.g., knockout animals).

Also provided herein are methods of treating a TREM2-related disease or disorder in a subject (e.g., a human) by administering to the subject a nucleic acid encoding a human TREM2 mutants resistant to sheddase cleavage as disclosed herein, or a vector or a cell comprising such a nucleic acid.

In some embodiments, the TREM2-related disease or disorder is a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis.

In some embodiments, the TREM2-related disease or disorder include CNS related diseases, PNS related diseases, systemic inflammation and other diseases related to inflammation, pain and withdrawal symptoms caused by an abuse of chemical substances, diseases or disorders related to the CNS include general anxiety disorders, cognitive disorders, learning and memory deficits and dysfunctions, Alzheimer's disease (mild, moderate and severe), attention deficit and hyperactivity disorder, Parkinson's disease, dementia in Parkinson's disease, Huntington's disease, ALS, prionic neurodegenerative disorders such as Creutzfeld-Jacob disease and kuru disease, Gilles de la Tourette's syndrome, psychosis, depression and depressive disorders, mania, manic depression, schizophrenia, the cognitive deficits in schizophrenia, obsessive compulsive disorders, panic disorders, eating disorders, narcolepsy, nociception, AIDS-dementia, senile dementia, mild cognitive impairment related to age (MCI), age associated memory impairment, autism, dyslexia, tardive dyskinesia, epilepsy, and convulsive disorders, post-traumatic stress disorders, transient anoxia, pseudodementia, pre-menstrual syndrome, late luteal phase syndrome, chronic fatigue syndrome and jet lag.

The TREM2-related disease or disorder also include: immunological disorders, especially involving inflammatory disorders (e.g., bacterial infection, fungal infection, viral infection, protozoa or other parasitic infection, psoriasis, septicemia, cerebral malaria, inflammatory bowel disease, arthritis, such as rheumatoid arthritis, folliculitis, impetigo, granulomas, lipoid pneumonias, vasculitis, and osteoarthritis), autoimmune disorders (e.g., rheumatoid arthritis, thyroiditis, such as Hashimoto's thyroiditis and Graves' disease, insulin-resistant diabetes, pernicious anemia, Addison's disease, pemphigus, vitiligo, ulcerative colitis, systemic lupus erythematosus (SLE), Sjogren's syndrome, multiple sclerosis, dermatomyositis, mixed connective tissue disease, scleroderma, polymyositis, graft rejection, such as allograft rejection), T cell disorders (e.g., AIDS), allergic inflammatory disorders (e.g., skin and/or mucosal allergies, such as allergic rhinitis, asthma, psoriasis), neurological disorders, eye disorders, embryonic disorders, or any other disorders (e.g., tumors, cancers, leukemia, myeloid diseases, and traumas) which are directly or indirectly associated with aberrant TREM2 activity and/or expression.

In some embodiments, the TREM2-related disease or disorder is an autoimmune, inflammatory, or malignant disorder mediated by or associated with extensive proteolytic cleavage of TREM2 or cells expressing aberrant or mutated variants of the TREM2 receptor. Examples of autoimmune diseases include, without limitation, arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankolsing spondylitis, Reiter syndrome, reactive arthritis, psoriatic arthritis, and enterophathis arthritis, hypersensitivity (including both airways hypersensitivity and dermal hypersensitivity) and allergies. Autoimmune diseases include autoimmune haematological disorders (including e.g. hemolytic anaemia, aplastic anaemia, pure red cell anaemia and idiopathic thrombocytopenia), systemic lupus erythematosus, inflammatory muscle disorders, polychondritis, sclerodoma, Wegener granulomatosis, dermatomyositis, chronic active hepatitis, myasthenia gravis, psoriasis, Steven-Johnson syndrome, idiopathic sprue, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca andvernal keratoconjunctivitis, interstitial lung fibrosis, psoriatic arthritis and glomerulonephritis (with and without nephrotic syndrome, e.g. including gout, langerhans cell histiocytosis, idiopathic nephrotic syndrome or minimal change nephropathy), tumors, inflammatory disease of skin and cornea, myositis, loosening of bone implants, metabolic disorders, such as atherosclerosis, diabetes, and dislipidemia.

In some embodiments, the TREM2-related disease or disorder is selected from asthma, bronchitis, pneumoconiosis, pulmonary emphysema, other obstructive or inflammatory diseases of the airways including idiopathic pulmonary fibrosis or COPD.

In some embodiments, the TREM2-related disease or disorder is a hematopoietic or hepatopoetic malignant disorder such as acute myeloid leukemia, chronic myeloid leukemia, myeloproliferative disorders, myelodysplastic syndromes, multiple myeloma, paroxysmal nocturnal hemoglobinuria, fanconi anemi, thalassemia major, Wiskott-Aldrich syndrome, hemophagocytic lymphohistiocytosis.

In some embodiments, the TREM2-related disease or disorder is selected from asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, or chronic inflammation resulting from chronic viral or bacterial infections.

In some embodiments, the TREM2-related disease or disorder is selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, Taupathy disease, Nasu-Hakola disease, stroke, acute trauma, chronic trauma, lupus, acute and chronic colitis, wound healing, Crohn's disease, inflammatory bowel disease, ulcerative colitis, obesity, Malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer.

In some embodiments, the TREM2-related disease or disorder is selected from dementia, frontotemporal dementia, Alzheimer's disease, Nasu-Hakola disease, and multiple sclerosis. In some embodiments, the TREM2-related disease or disorder is a dementia such as frontotemporal dementia, Alzheimer's disease, vascular dementia, semantic dementia, or dementia with Lewy bodies. In some embodiments, the TREM2-related disease or disorder is Alzheimer's disease. In some embodiments, the TREM2-related disease or disorder is frontotemporal dementia.

In some embodiments, the nucleic acid, vector, or cell is administered to the subject through an intravenous, intracranial, intrathecal, subcutaneous, or intranasal route.

In some embodiments, such methods also include assaying the cell surface human TREM2 level in a sample obtained from a subject, e.g., a cerebrospinal fluid sample. The level of cell surface human TREM2 in the sample can be determined by an assay known in the art, e.g., by flow cytometry, immunohistochemistry, Western blotting, immunofluorescent assay, radioimmunoassay (RIA), enzyme-linked immunosorbent assay (ELISA), homogeneous time resolved fluorescence (HTRF), or positron emission tomography (PET), or any other immune detection with an antibody or antibody fragment against human TREM2 protein.

Provided herein are also uses of a nucleic acid encoding a human TREM2 mutants resistant to sheddase cleavage as disclosed herein, or a vector or a cell comprising such a nucleic acid, for treatment of a TREM2-related disease or disorder in a subject. Uses of a nucleic acid encoding a human TREM2 mutants resistant to sheddase cleavage as disclosed herein, or a vector or a cell comprising such a nucleic acid, in the manufacture of a medicament for treatment of a TREM2-related disease or disorder are also included.

### Combination Therapies

The various treatments described above can be combined with other treatment partners such as the current standard of care for a TREM2-related disease or disorder, e.g., the current standard of care for Alzheimer's disease, frontotemporal dementia, Parkinson's disease, amyotrophic lateral sclerosis, or Nasu-Hakola disease. For example, nucleic acids encoding a human TREM2 mutant resistant to sheddase cleavage as described herein or vectors or cells containing such nucleic acids can be combined with one or more of BACE inhibitors, anti-Tau antibodies, anti-amyloid beta antibodies, FTY720, BG12, interferon beta or tysabri. Accordingly, the methods of treating a TREM2 related disease or disorder described herein can further include administering a second agent to the subject in need of treatment.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g., a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Sample Preparation

Samples used in the methods described herein can be obtained from a subject using any of the methods known in the art, e.g., by biopsy or surgery. For example, a sample comprising cerebrospinal fluid can be obtained by lumbar puncture, in which a fine needle attached to a syringe is inserted into the spinal canal in the lumbar area and a vacuum is created such that cerebrospinal fluid may be sucked through the needle and collected in the syringe. CT imaging, ultrasound, or an endoscope can be used to guide this type of procedure. The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

### Pharmaceutical Compositions, Dosage, and Methods of Administration

Also provided herein are compositions, e.g., pharmaceutical compositions, comprising one or more nucleic acids encoding a human TREM2 mutant resistant to sheddase cleavage as described herein, or vectors or cells containing such nucleic acids. Such compositions can further include another agent, e.g., a current standard of care for the disease to be treated.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral (e.g., intravenous, intraarterial, intraperitoneal), oral, intracranial, intrathecal, or intranasal (e.g., inhalation), intradermal, subcutaneous, or transmucosal administration. In some embodiments, the pharmaceutical compositions are formulated to deliver TREM2-binding molecules to cross the blood-brain barrier.

In some embodiments, the pharmaceutical compositions comprise one or more pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Methods of formulating suitable pharmaceutical compositions are known in the art, see, e.g., Remington: The Science and Practice of Pharmacy. 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders, for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Parenteral formulations can be a single bolus dose, an infusion or a loading bolus dose followed with a maintenance dose. These compositions can be administered at specific fixed or variable intervals, e.g., once a day, or on an “as needed” basis.

A suitable pharmaceutical composition for injection can comprise a buffer (e.g., acetate, phosphate or citrate buffer), a surfactant (e.g., polysorbate), optionally a stabilizer agent (e.g., human albumin), etc. Preparations for peripheral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In some embodiments, the pharmaceutical composition comprises 0.01-0.1 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems.

The pharmaceutical compositions can be included in a container, pack, or dispenser together with instructions for administration.

In non-limiting examples, the pharmaceutical composition containing at least one pharmaceutical agent is formulated as a liquid (e.g., a thermosetting liquid), as a component of a solid (e.g., a powder or a biodegradable biocompatible polymer (e.g., a cationic biodegradable biocompatible polymer)), or as a component of a gel (e.g., a biodegradable biocompatible polymer). In some embodiments, the at least composition containing at least one pharmaceutical agent is formulated as a gel selected from the group of an alginate gel (e.g., sodium alginate), a cellulose-based gel (e.g., carboxymethyl cellulose or carboxyethyl cellulose), or a chitosan-based gel (e.g., chitosan glycerophosphate). Additional, non-limiting examples of drug-eluting polymers that can be used to formulate any of the pharmaceutical compositions described herein include, carrageenan, carboxymethylcellulose, hydroxypropylcellulose, dextran in combination with polyvinyl alcohol, dextran in combination with polyacrylic acid, polygalacturonic acid, galacturonic polysaccharide, polysalactic acid, polyglycolic acid, tamarind gum, xanthum gum, cellulose gum, guar gum (carboxymethyl guar), pectin, polyacrylic acid, polymethacrylic acid, N-isopropylpolyacrylomide, polyoxyethylene, polyoxypropylene, pluronic acid, polylactic acid, cyclodextrin, cycloamylose, resilin, polybutadiene, N-(2-Hydroxypropyl)methacrylamide (HP MA) copolymer, maleic anhydrate - alkyl vinyl ether, polydepsipeptide, polyhydroxybutyrate, polycaprolactone, polydioxanone, polyethylene glycol, polyorganophosphazene, polyortho ester, polyvinylpyrrolidone, polylactic-co-glycolic acid (PLGA), polyanhydrides, polysilamine, poly N-vinyl caprolactam, and gellan.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method disclosed herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Kits

Also provided herein are kits including one or more nucleic acids encoding a human TREM2 mutant resistant to sheddase cleavage as described herein or vectors or cells containing such nucleic acids and instructions for use. Instructions for use can include instructions for diagnosis or treatment of a TREM2-related disease or disorder. Kits as provided herein can be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

One skilled in the art will recognize many methods and materials similar or equivalent to those described herein, which could be used in the practice of the present invention. Indeed, the present invention is in no way limited to the methods and materials described.

### EXAMPLES

The invention is further described in the following examples, which do not limit the scope of the invention described in the claims.

### Example 1: Materials and Methods

### Compounds

GI254023 ((2R,3 S)-3 -(formyl-hydroxyamino)-2-(3-phenyl-1-propyl) butanoic acid [(1S)-2,2-dimethyl-1-methylcarbamoyl-1-propyl] amide) was synthesized as described in Hundhausen et al., Blood. 2003; 102:1186-1195). DPC333 ((2R)-2-((3R)-3-amino-3{4-[2-methyl-4-quinolinyl) methoxy]phenyl}-2-oxopyrrolidinyl)-N-hydroxy-4-methylpentanamide)) was synthesized as described in Qian et al., Drug metabolism and disposition: the biological fate of chemicals 35, 1916-1925, 2007.

### Cell Culture

THP1 cells stably coexpressing Cas9 and a blasticidin resistance gene delivered by lentivirus were cultured in RPMI medium containing 10% FBS, 1% L-glutamine, 1% pen/strep, and 10 μg/ml of blasticidin (Thermo Fisher Scientific). The cells were cultured at 37° C. in 5% CO₂ atmosphere.

### Generation of ADAM17 and ADAM10 Knockout Lines

THP1-Cas9 cells were infected with lentiviruses expressing the puromycin resistance gene and sgRNAs (for vector design, see Hoffman, Proceedings of the National Academy of Sciences of the United States of America 111, 3128-3133, 2014) targeting either ADAM10 (GTAATGTGAGAGACTTTGGG, SEQ ID NO: 75) or ADAM17 (CCGAAGCCCGGGTCATCCGG, SEQ ID NO: 76). Lentiviral packaging was carried out in HEK293T cells as described previously. Briefly, 30 μL of lentiviral sgRNA supernatant was added to 1×10⁶ THP1-Cas9 cells in 2 ml medium containing 5 μg/ml polybrene (Sigma) and spun at 300 g for 90 minutes in a 6-well plate. After 24 hours the cells were spun down and resuspended in fresh culture medium containing 1.5 μg/mL puromycin. After 4 weeks of weekly media changes, genomic DNA was isolated using a Quick-gDNA miniprep kit (Zymo Research) to assess the insertions and deletions (indels) present in the pool by next-generation sequencing (NGS). To isolate clones containing only frameshift indels, cells were plated at limiting dilution into 96-well plates. Upon expansion of the clones, they were assayed by NGS and clones containing only frameshift alleles were selected for downstream assays.

### NGS Indel Analysis

To prepare engineered cells for NGS, each target was amplified using locus-specific primers. Two rounds of PCR were performed. The first round utilized locus-specific primers to amplify the edited region. The primers for ADAM10 were ATTAGACAATACTTACTGGGGATCC (SEQ ID NO: 77) and GGAAGCTCTGGAGGAATATGTG (SEQ ID NO: 78), and the primers for ADAM17 were CCCCCAAACACCTGATAGAC (SEQ ID NO: 79) and CCAGAGAGGTGGAGTCGGTA (SEQ ID NO: 80). The product formed during the first round was then used as a template for a second round of PCR to add dual indices compatible with the Illumina system. The Illumina Nextera Adapter sequences for both target regions were TCGTCGGCAGCGTCAGATGTGTATAAGAGACAG (SEQ ID NO: 81) and GTCTCGTGGGCTCGGAGATGTGTATAAGAGACAG (SEQ ID NO: 82). Libraries were quantitated by qRT-PCR and subsequently sequenced on the Illumina MiSeq system. For sequence analysis, raw reads were aligned to a reference sequence, then tallied based on genotype. Finally, tallied genotypes were binned into one of three categories: wild-type, in-frame, and frameshift.

### Cell Surface Expression of Mutated TREM2 in HEK293 Cells

Human TREM2 mutants were generated using the QuikChange Site-directed Mutagenesis Kit (Stratagene) and confirmed by sequence analysis. Transfections of complementary DNA (cDNA) constructs were carried out in a 1:1 ratio of hDAP12 and TREM2 in HEK-FT cells using Lipofectamine LTX reagent (Thermofischer) according to the manufacturer's recommendations. 48 h after transfection cells were treated for 30 min with 50 ng/ml PMA or 0.05% DMSO and detached with a cell detachment solution Accutase (Sigma) and stained with goat anti human TREM2 antibody AF1828 (R&D Systems) or isotype control followed by incubation with the secondary Alexa Fluor 488 conjugated antibody (Molecular Probes). Acquisition was performed using a BD FACSCanto II (BD biosciences).

### TREM2 Expression in Human Macrophages and CHO Cells

CHO cells were transfected to co-express hDAP12 and hTREM2 using Lipofectamine LTX reagent (Thermofischer) according to the manufacturer's recommendations. One positive clone was selected and designated CHO-hDAP12-hTREM2. Human M2A macrophages were obtained from buffy coat using a negative isolation kit for monocytes (Stem cell technologies) and differentiated for 5 days in RPMI1640 medium with Glutamax (Gibco) supplemented with 10% FBS (Gibco), PenStrep (Gibco), 1% Sodium Pyruvate (Gibco), 0.025 M HEPES Buffer (Gibco), 0.05 mM β-mercaptoethanol (Gibco), M-CSF (40 ng/ml) and IL-4 (50 ng/ml). Cell surface TREM2 was detected by FACS as described above.

### Live Cell Imaging

Human M2A macrophages or CHO-hDAP12-hTREM2 were seeded on 384 well plates (Greiner) and treated with ADAM inhibitors DPC333 or GI254023 at concentrations indicated in the figures. 16 h later cells were treated for 30 minutes with PMA (50 ng/ml) or 0.1% DMSO for 30 minutes. Plates were put on ice and stained with goat anti human TREM2 antibody AF1828 (R&D Systems) or isotype control and Hoechst stain followed by incubation with the secondary Alexa Fluor 488 conjugated anti goat antibody (Molecular Probes). Images were acquired using an InCe112000 analyzer (GE Healthcare). For image quantification the free open source software CellProfiler was applied.

### Reporter Gene Assay in BWZ Cells

BWZ thymoma reporter cells, which express lacZ under control of the promoter for nuclear factor of activated T cells (NFAT, Hsieh et al, Journal of Neurochemistry 109, 1144-1156, 2009) were transfected to co-express mDAP12 and WT hTREM2 or T2double TREM2. Cells were seeded in RPMI without phenol red supplemented with 2% FBS and 1% non-essential amino acids on high binding microtiter plates (Greiner) pre-coated with rat anti-mouse/human TREM2 mAb (R&D, MAB17291) or isotype control. Cell culture was continued for 16 h. Reporter gene activity was assessed with the Beta-glo assay system (Promega) according to the manufacturer's recommendations using an Envision 2104 multilabel reader (Perkin Elmer).

### Immuno-Purification

Shed TREM2 was purified from cell supernatant through microscale immuno-purification. This was performed on the MEA platform (PhyNexus) and Streptavidin coated tips (PTR 92-05-05, Phynexus) have been used. First the tips are equilibrated with PBS, then 200 μL of biotinylated anti-TREM2 antibody (0.55 pmol/μL, BAF1828 from R&D System), is loaded onto the streptavidin μ column (5 μL, bed volume) at a speed of 0.25 mL/min and 8 passages. After a wash with PBS, the shed TREM2 is captured from the cell supernatant (200 μL) at a speed of 25 mL/min and 12 passages. It is followed by PBS wash and elution by 0.1M Glycine pH 2.5 (2×4 passages) for a final volume of 2×60 μL. The latter solution is neutralized with the addition of 1 M Tris-HCl pH 10 (5 μl), then it was dried (Speedvac) and rehydrated with 8M urea (5 μL, Fluka) and 0.4M NH₄CO₃ (30 μL, Fluka). The sample is then reduced (2 μL of 1M DTT, 30 min at 50° C.), alkylated (6 μl of 1M IAA (Sigma), 30 min at RT in dark) and the reaction was terminated with the addition of 1M DTT (2 μL) and 0.4M NH₄CO₃ (30 μl). The resulting sample is either digested by Trypsin or Asp-/Glu-C enzyme (+1 μl of Trypsin (Promega) or Asp-/Glu C (Roche), 1 μg/μl, pH 8, and overnight incubation at 37° C.). The digested sample is finally acidified with HCOOH (1 μL, Fluka) and 25 μL of the resulting digest were injected onto the LC-MS platform.

### Mass Spectrometry

Identification of the cleavage site by peptide mapping: the LC-MSE analyses were performed using a SYNAPT G2S QTOF mass spectrometer (WATERS) coupled with an UPLC (ACQUITY I class, WATERS). A BEH C18 UPLC column (1.7 μm, 1×100 mm, WATERS) was used for peptide separation. An elution gradient with mobile phase A (0.1% HCOOH in water) and mobile phase B (0.1% HCOOH in acetonitrile) was generated using the following program: 1) isocratic at 2% B for 3 min; 2) linear gradient from 2 to 30% B from 3 to 90 min; 3) linear gradient from 30 to 100% B from 90 to 95 min; 4) isocratic at 100% B from 95 to 105 min; 5) linear gradient from 100 to 2% B from 105 to 105.5 min; and finally 6) isocratic at 2% B from 105.5 to 120 min. The mass spectrometer was working in positive resolution mode with automatic mass correction through a lockspray system (P14R peptide, m/z 767.433, infused at 250 fmol at 5 μL/min, switch frequency was every 20 sec for 0.5 sec per scan, 3 scans averaged). 2 MS traces were acquired, one MS and one for MSE. Both were acquired in mass range m/z 50-2000, scan time 0.5 sec, 3 kV capillary voltage, 40 V cone voltage. In MSE mode the trap voltage was ramped in each scan from 20 to 40 V. In addition, a UV trace was acquired at a wavelength of 214 nm.

### Identification of the Cleavage Site by Intact Mass Measurement

The LC-MS analyses were performed using a SYNAPT G1 QTOF mass spectrometer (WATERS) coupled with an UPLC (ACQUITY I class, WATERS). A BEH C4 UPLC column (1.7 μm, 1×100 mm, WATERS) was used for protein separation. An elution gradient with mobile phase A (0.1% HCOOH in water) and mobile phase B (0.1% HCOOH in acetonitrile) was generated using the following program: 1) isocratic at 5% B for 1.5 min; 2) linear gradient from 5 to 25% B from 1.5 to 2 min; 3) linear gradient from 25 to 35% B from 2 to 12 min; 4) linear gradient from 35 to 95% B from 12 to 13 min; 5) isocratic at 95% B from 13 to 15 min; 5) linear gradient from 95 to 5% B from 15 to 15.5 min; and finally 6) isocratic at 5% B from 15.5 to 20 min. The mass spectrometer was working in positive resolution mode and calibrated with NaI 2 mg/mL. MS trace was acquired in mass range m/z 600-4500, scan time 0.5 sec, 3 kV capillary voltage, 40 V cone voltage, desolvation temperature 200° C., cone gas flow 50 L/h. In addition, a UV trace was acquired at a wavelength of 214 nm.

### Identification of O-Linked Glycosylation

The LC-MSE analyses were performed using a QTOF Premier mass spectrometer (WATERS) coupled with an UPLC (ACQUITY H class, WATERS). A BEH C18 UPLC column (1.7 μm, 1×100 mm, WATERS) was used for peptide separation. An elution gradient with mobile phase A (0.1% HCOOH in 98% water and 2% acetonitrile) and mobile phase B (0.1% HCOOH in acetonitrile) was generated using the following program: 1) isocratic at 2% B for 3 min; 2) linear gradient from 2 to 30% B from 3 to 90 min; 3) linear gradient from 30 to 100% B from 90 to 95 min; 4) isocratic at 100% B from 95 to 105 min; 5) linear gradient from 100 to 2% B from 105 to 105.5 min; and finally 6) isocratic at 2% B from 105.5 to 120 min. The mass spectrometer was working in positive normal mode with a lockspray system (P14R peptide, infused at 1 pmol at 10 μL/min, switch frequency was every 20 sec for 0.5 sec per scan, 3 scans averaged). Mass correction was performed by application of a lockmass (2+, 767.433 Da) during data processing with PLGS (WATERS). 2 MS traces were acquired, one MS and one for MSE. Both were acquired in mass range m/z 50-2000, scan time 0.5 sec, 3 kV capillary voltage, 40 V cone voltage. In MSE mode the trap voltage was ramped in each scan from 20 to 40 V. In addition, a UV trace was acquired at a wavelength of 214 nm.

### Statistical Analysis

Statistical analysis was performed using Prism software (GraphPad, San Diego, Calif.) using ANOVA and student's t test where appropriate. A p value of <0.05 was considered significant.

### Example 2: ADAM17 Inhibitors Stabilize TREM2 at the Cell Surface

Triggering receptor expressed in myeloid cells (TREM2) is a type I transmembrane glycoprotein and a member of the immunoglobulin (Ig) receptor superfamily (Bouchon et al., The Journal of Experimental Medicine 194, 1111-1122, 2001). TREM2 expression has been shown in macrophages, dendritic cells, microglia and osteoclasts, and expression seems to be temporally and spatially regulated (Lue et al., Neuroscience 302, 138-150, 2015; Schmid et al., Journal of Neurochemistry 83, 1309-1320, 2002; Sessa et al., The European Journal of Neuroscience 20, 2617-2628, 2004). In macrophages, TREM2 expression is upregulated during the course of inflammation, e.g. expression peaks 2-3 days after thioglycolate challenge in a murine model of peritonitis (Turnbull et al., Journal of Immunology 177, 3520-3524, 2006). TREM2 is also enriched at those microglia cell surface regions which contact Aβ plaques or neuronal debris (Yuan et al., Neuron 90, 724-739, 2016). Some of the ligands that are sensed by TREM2 in this environment have recently been identified, for example phospholipids and myelin lipids (Poliani et al., The Journal of Clinical Investigation 125, 2161-2170, 2015) as well as ApoE (Atagi et al., The Journal of Biological Chemistry 290, 26043-26050, 2015; Bailey et al., The Journal of Biological Chemistry 290, 26033-26042, 2015). Other ligands could be Aβ and plaque associated neuronal debris since TREM2 contributes to the uptake of Aβ into microglia (Xiang et al., EMBO Molecular Medicine 8, 992-1004, 2016).

This is well in line with earlier genome wide association studies showing that a TREM2 SNP, rs75932628-T encoding the R47H variant, conferred a significantly increased risk of late onset Alzheimer's disease (LOAD) with odds ratios of 5.05 (Guerreiro et al., The New England Journal of Medicine 368, 117-127, 2013) and 2.92 (Jonsson et al., The New England Journal of Medicine 368, 107-116, 2013). These odds ratios are comparable with that of the well-established AD risk gene APOE4 (Neumann & Daly, The New England Journal of Medicine 368, 182-184, 2013). It is of note that R47H mutated TREM2 displays almost the same cell surface expression as WT TREM2 (Kleinberger, 2014) but is functionally impaired: the R47H mutation in TREM2 reduces binding of lipid ligands (Wang et al., Cell 160, 1061-1071, 2015) and ApoE (Atagi, 2015; Bailey, 2015). The mutation also reduces phagocytic capacity (Kleinberger, 2014) and impedes recycling of TREM2 via Vps35 within the retromer complex (Yin et al., Traffic 17, 1286-1296, 2016). Some human R47H carriers without AD have been characterized, and these individuals lose brain volume faster than non-carriers (Rajagopalan et al., The New England Journal of Medicine 369, 1565-1567, 2013), have a poorer cognitive function than age matched controls, (Jonsson et al., The New England Journal of Medicine 368, 107-116, 2013) and show upregulation of pro-inflammatory cytokines (e.g. RANTES, INFγ) and downregulation of protective markers (e.g. IL-4, ApoA1; See Roussos et al., Alzheimer's & Dementia: the Journal of the Alzheimer's Association 11, 1163-1170, 2015).

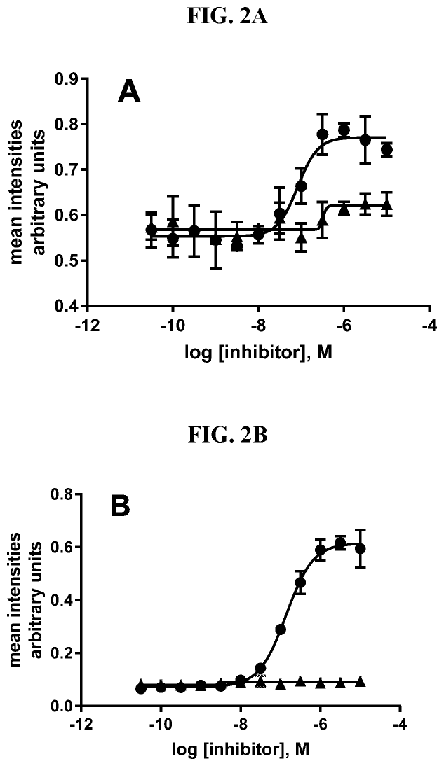

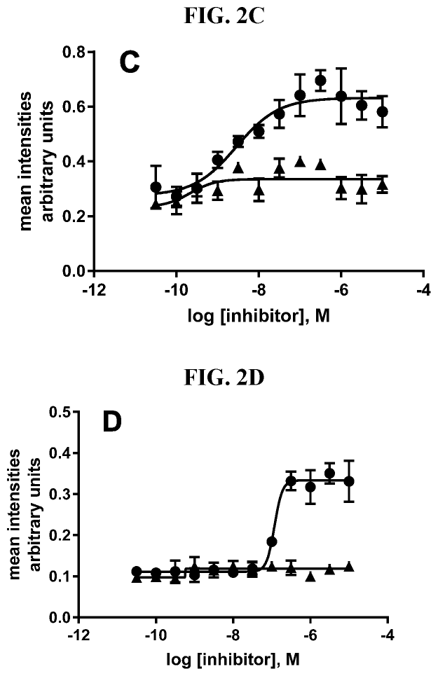

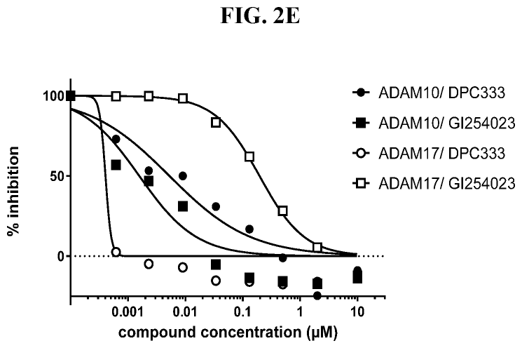

To determine the contribution of ADAM10 or ADAM17 to shedding of TREM2 ectodomain, selective inhibitors were identified: DPC333 (Qian et al., Drug Metabolism and Disposition: the Biological Fate of Chemicals 35, 1916-1925, 2007) and GI254023 (Hundhausen et al., Blood. 2003; 102:1186-1195). DPC333 and GI254023 were characterized for inhibitory selectivity towards ADAM10 and ADAM17. FIG. 2E showed DPC333 is a more potent inhibitor on ADAM17 (IC₅₀<0.6 nM) than on ADAM10 (IC₅₀=5.3 nM), and GI254023 displays selectivity for ADAM10 (IC₅₀ 1.5 nM) over ADAM17 (IC₅₀=196 nM). Using live cell imaging, cell surface expression of hTREM2 was assessed in CHO-hDAP12-hTREM2 cells after overnight treatment of the cells with the two ADAM inhibitors under conditional shedding conditions (FIG. 2A) or after treatment of the cells with PMA (FIG. 2B). The ADAM17 selective inhibitor DPC333 dose-dependently increases TREM2 cell surface levels under both conditions. A limited effect on TREM2 cell surface levels is also observed at higher concentrations for GI254023, but only under steady state conditions. This effect might be attributed to true ADAM10 inhibition or it might be caused by unspecific inhibition of ADAM17 by GI254023 when used at high concentrations. In PMA-treated cells there is a complete lack of effect of GI254023 on TREM2 cell surface expression (FIG. 2B). To get closer to a physiological cellular system, a similar experiment was conducted in human M2A macrophages differentiated from CD¹⁴⁺ human monocytes (FIGS. 2C-2D). These results replicate very well the initial findings in CHO-hDAP12-hTREM2 cells; the ADAM17 inhibitor DPC333 increases TREM2 cell surface expression dose-dependently under both conditions (FIGS. 2C-2D) and the selective ADAM10 inhibitor displays a small effect on steady-state shedding (FIG. 2C).

In summary, these experiments indicate that in human macrophages, ADAM17 plays a critical role for TREM2 shedding but a marginal contribution of ADAM10 under steady state condition cannot be excluded.

### Example 3: ADAM17 Ablation in THP1 Cells Reduces Constitutive Shedding

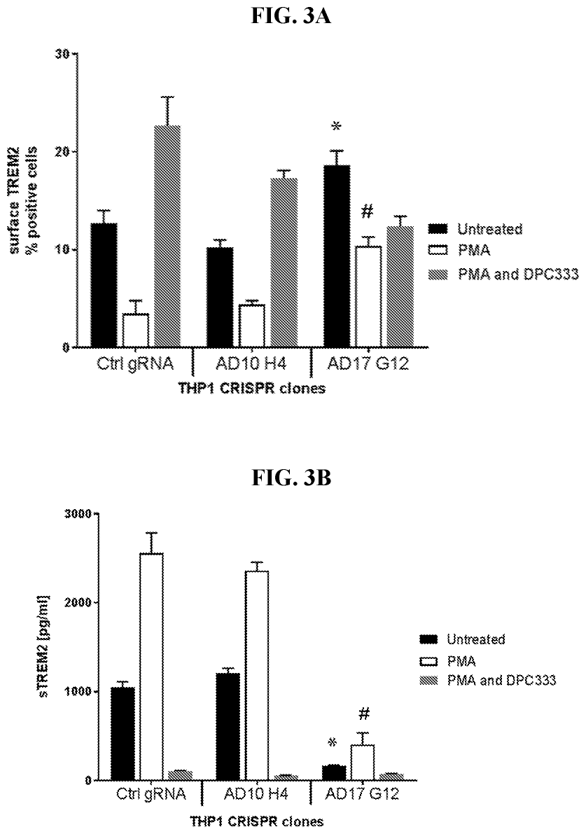

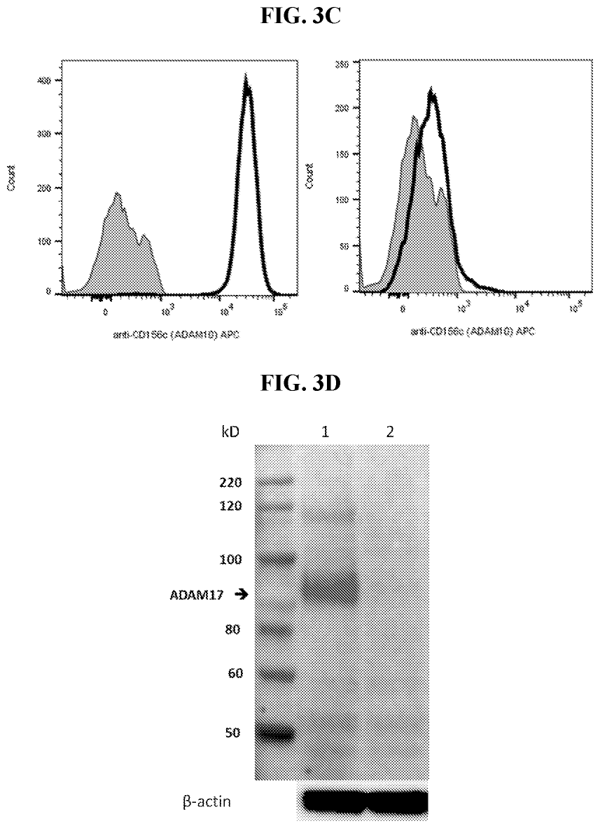

To confirm the results in Example 2, a genetic approach was used to further investigate the contribution of ADAM10/17 to TREM2 shedding. Human monocytic THP1 cells were chosen as model system which endogenously expresses TREM2. Employing the CRISPR/CAS9 technology, clones were generated that lack expression of ADAM10 (AD10 H4) or ADAM17 (AD17 G12) as well as a control cell line (Ctrl gRNA). Absence of gene products was verified by FACS analysis or Western blot (FIGS. 3C-3D).

Cell surface expressed TREM2 and sTREM2 were assessed in the three cell lines in the same experiment using three different conditions: no treatment reflecting constitutive shedding, PMA treatment that maximally activates shedding, and finally PMA and DPC333 treatment. Loss of ADAM17 increases TREM2 cell surface expression and strongly reduces soluble TREM2 under conditional shedding conditions, whereas lack of ADAM10 has no significant effects (see black bars in FIGS. 3A-3B), thus indicating that ADAM17 is the main sheddase contributing to constitutive shedding. Maximally activating sheddases with PMA leads to a strong reduction of cell surface TREM2 both in the control cell line and in the ADAM10 deficient clone. This is reflected in a strong increase in sTREM2. In the ADAM17 deficient cell line, PMA treatment also leads to a reduction of cell surface TREM2, but to a smaller extent than in the control CRISPR clone. Also, the increase in sTREM2 is smaller compared to PMA treated control CRISPR clone and ADAM10 deficient clone. However, the cleavage of TREM2 in the AD17 G12 clone in the presence of PMA must be caused by a sheddase other than ADAM17. Accordingly, in the ADAM10 H4 clone the increase in PMA induced shedding might be caused by additional activation of ADAM17. Co-treatment with PMA and DPC333 restored TREM2 cell surface levels in Ctrl gRNA and AD10 H4 cells but had smaller effects in the AD17 G12 clone. In the AD17 G12 clone cell surface TREM2 levels do not reach the extent that is seen under constitutive shedding conditions. sTREM2 however is strongly reduced in the AD17 G12 clone under these conditions compared to PMA treatment only.

In summary, in THP1 cells, ADAM17 seems to be the main sheddase responsible for constitutive shedding. After PMA treatment additional shedding mechanisms come into play, one of which might involve ADAM10.

### Example 4: The Amino Acid Stretch Close to the TREM2 Transmembrane Domain are Important for Shedding

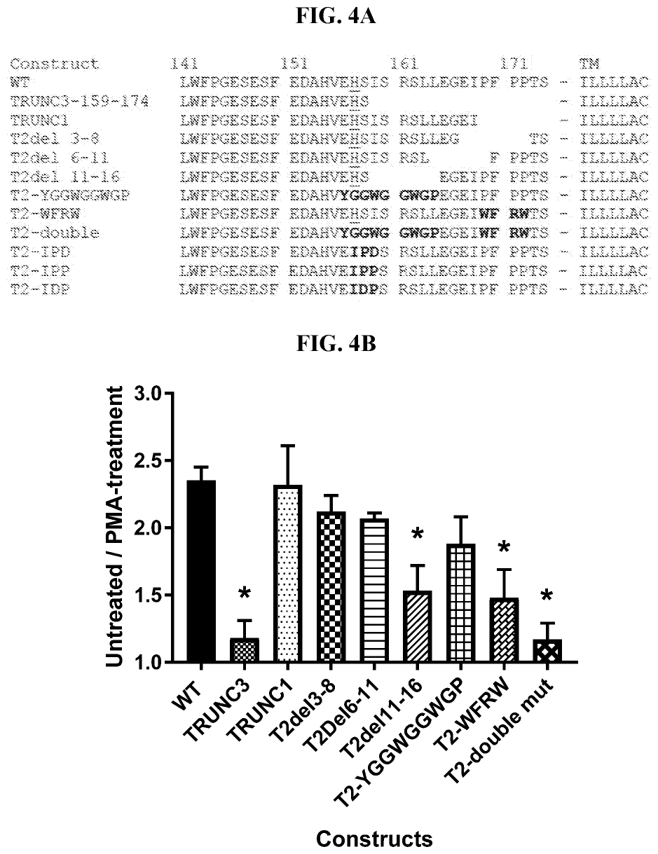

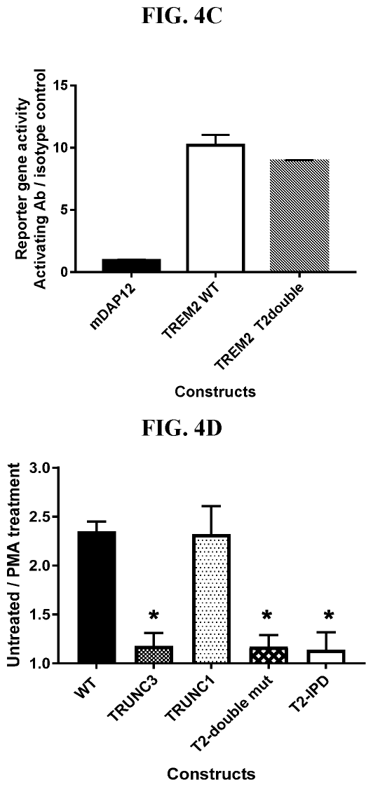

In the next experiments, site-directed mutagenesis was used to identify areas within TREM2 stalk region that harbor the cleavage site or are important for binding of the sheddases. FIG. 4A shows the different TREM2 mutants that have been generated and tested. Table 4 lists the amino acid sequences of the membrane proximal part of the wild type or mutant human TREM2 stalk region and transmembrane domain.

TABLE 4
Amino acid sequence of the membrane proximal part of the wild type or
mutant human TREM2 stalk region and transmembrane domain

| | SEQ | |
|---|---|---|
| Name | ID NO | Sequence |
| WT | 12 | LWFPGESESFEDAHVEHSISRSLLEGEIPFPPTSILLLLAC |
| TRUNC3 (159- | 13 | LWFPGESESFEDAHVEHSILLLLAC |
| 174 deletion) | | |
| TRUNC1 | 14 | LWFPGESESFEDAHVEHSISRSLLEGEIILLLLAC |
| T2del 3-8 | 15 | LWFPGESESFEDAHVEHSISRSLLEGTSILLLLAC |
| T2del 6-11 | 16 | LWFPGESESFEDAHVEHSISRSLFPPTSILLLLAC |
| T2del 11-16 | 17 | LWFPGESESFEDAHVEHSEGEIPFPPTSILLLLAC |
| T2-YGG | 18 | LWFPGESESFEDAHVYGGWGGWGPEGEIPFPPTSILLLLAC |
| T2-WFR | 19 | LWFPGESESFEDAHVEHSISRSLLEGEIWFRWTSILLLLAC |
| T2-double | 20 | LWFPGESESFEDAHVYGGWGGWGPEGEIWFRWTSILLLLAC |
| T2-IPD | 21 | LWFPGESESFEDAHVEIPDSRSLLEGEIPFPPTSILLLLAC |
| T2-IPP | 22 | LWFPGESESFEDAHVEIPPSRSLLEGEIPFPPTSILLLLAC |
| T2-IDP | 23 | LWFPGESESFEDAHVEIDPSRSLLEGEIPFPPTSILLLLAC |

Wild-type (WT) TREM2 or TREM2 mutants were transfected together with hDAP12 into HEK-FT cells. 48 h later cells were treated for 30 min with PMA to activate ADAMs at cell surface (see Sommer, Nature Communications 7, 11523, 2016). TREM2 cell surface expression was assessed by FACS, and results are presented as expression ratio of untreated over PMA treatment (FIG. 4B). The evaluation of each construct in the presence and absence of PMA treatment overcomes the possibility that some constructs might display different binding properties for the antiserum and allows direct comparison of changes in TREM2 cell surface expression after activation of sheddases. A ratio of 1 indicates complete inhibition of shedding. Deletion of the first 16 amino acids (AA) proximal to the transmemebrance domain (TM) maximally reduces TREM2 shedding (TRUNCIII-159-174). This suggests that this region might entail the cleavage and/or the binding site for ADAMs. Next four shorter deletion mutants were generated encompassing this area, each 6 amino acids long (TRUNC1, T2del3-8, T2del6-11 and T2del11-16). While there was no or very little effect on shedding for mutants TRUNC1, T2del3-8 and T2del6-11, the mutant T2del11-16 showed reduced PMA induced shedding (FIG. 4B).

Amino acid replacement mutants were designed to overcome the issue that deletion mutants might shift the cleavage site closer to the transmembrane region causing reduction of cleavage due to steric hindrance. Amino acids 156-164 and 169-172 were replaced with larger hydrophobic residues that should render the stalk region resistant to protease cleavage (Stromstedt et al., Antimicrobial agents and chemotherapy 53, 593-602, 2009). While T2-YGG mutant showed a trend for reduction of TREM2 shedding, replacement of 4 membrane-proximal amino acids T2-WFR mutant was more efficacious; and the combination of both mutations (T2-double mutant) had a similar effect as the deletion mutant TRUNCIII-159-174 (FIGS. 4A and 4B). Thus, these TREM2 mutants showed resistance to sheddase cleavage.

In summary, the mutagenesis approach revealed that two areas are important for PMA induced shedding of TREM2, a membrane proximal at amino acids 169-172 and a membrane distal in the region amino acids 156-164.

Next it was tested whether the T2-double-TREM2 construct retained functionality. To this end this mutant was stably transfected into BWZ cells that already expressed mouse DAP12 and a beta-Gal reporter driven by NFAT (Hsieh, Journal of Neurochemistry 109, 1144-1156, 2009). mDAP12 expressed without TREM2 in this cell lines represents background reporter gene activity (RGA) in this system. Comparing RGA after TREM2 activation in BWZ-T2-double and BWZ-TREM2-WT cells revealed comparable activity, indicating that mutated T2-double is still capable of signaling via DAP12 (FIG. 4C). Thus, the T2-double-TREM2 construct retained TREM2 functions.

### Example 5: ADAM17 Cleaves TREM2 Stalk Region Peptides at Position H157-S158

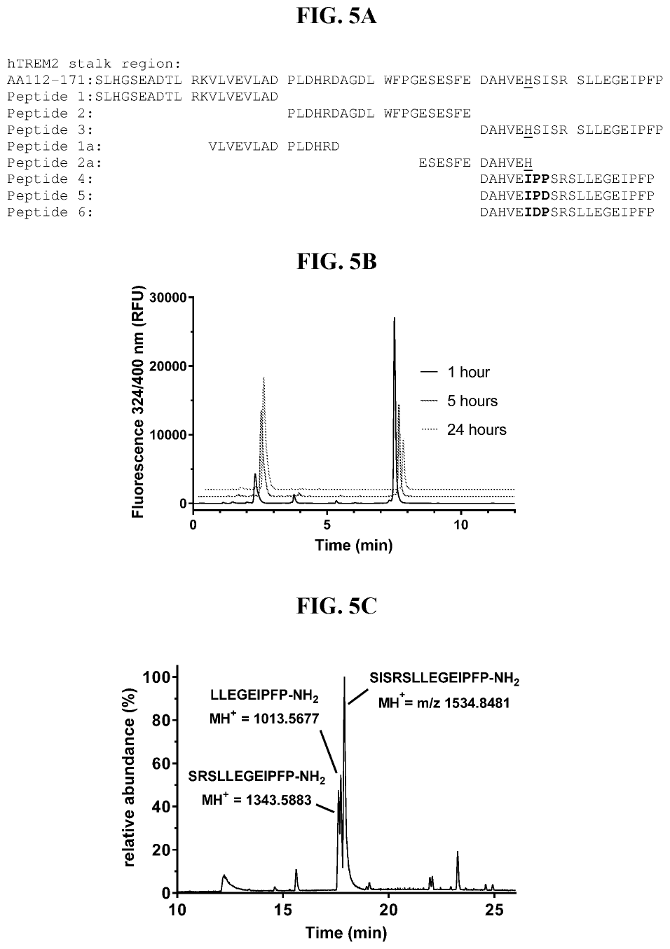

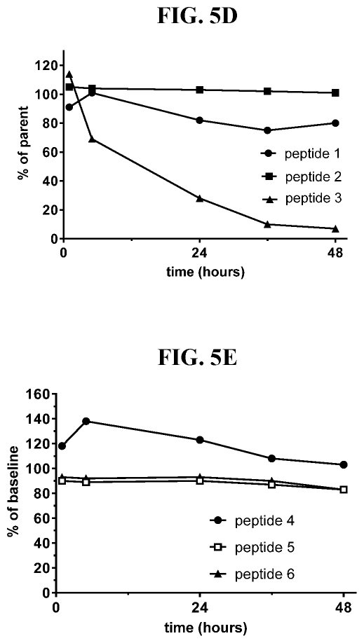

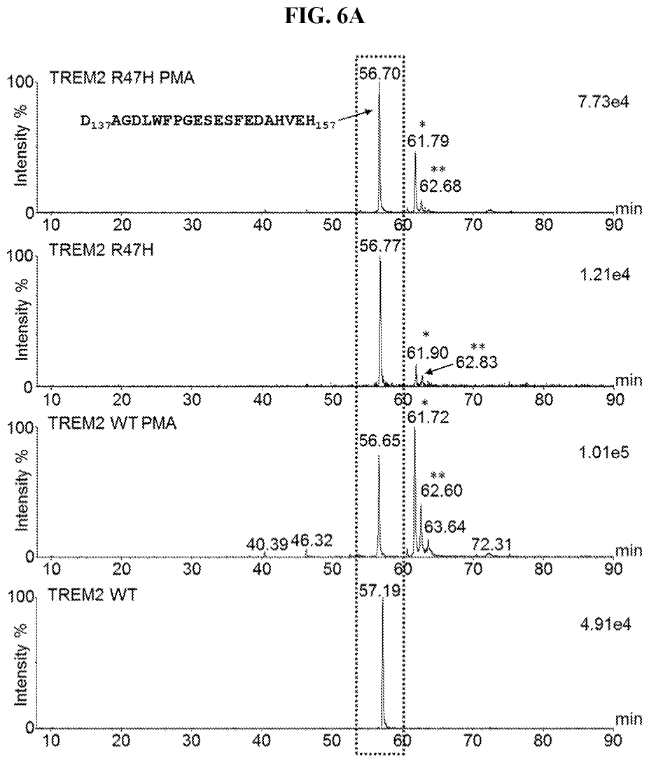

In order to identify the cleavage site of ADAM10/ADAM17 within the stalk region of TREM2, in vitro cleavage patterns of stalk region-derived peptides were investigated and confirmed the cleavage site by determination of the C-terminus of shed soluble TREM2 from cell culture supernatants. A series of peptides covering the stalk region were designed for in vitro investigation of ADAM10/ADAM17 cleavage (FIG. 5A). All peptides were obtained with an N-terminal 7-methoxycoumarin (Mca) fluorescent tag. Peptides 1-3, 1a and 2a were incubated with ADAM17 in neutral buffer for up to 48 hours, and reaction was followed by HPLC analysis of aliquots withdrawn at different time points. Significant cleavage was observed only for peptide 3, while peptides 1, 2, 1a, and 2a showed only very little or no reduction of the parent peptide (FIG. 5D).

HPLC-MS analysis of the reaction mixture of peptide3/ADAM17 identified one major product, SISRSLLEGEIPFP-NH2(SEQ ID NO: 51), together with 2 minor cleavage products (FIGS. 5B-5C). This suggests that the H157-S158 bond in TREM2 is the main cleavage site for ADAM17. HPLC analysis of the incubation mixture at times >24 hours showed appearance of more than one product peak, together with a reduced main product peak. At these time points there was essentially no substrate left. Therefore, it can be concluded that the minor cleavage products originate from secondary ADAM17 cleavage of the main product. The same analysis was carried out with ADAM10, and a very similar cleavage pattern was obtained (data not shown).

Recent literature gave insight into ADAM10 and ADAM17 preferences for the cleavage of peptides and proteins (Caescu et al., Biochem J 424, 79-88, 2009; Tucher et al., J Proteome Res 13, 2205-2214, 2014). Surprisingly, very rarely His was found in P1, and Ser was found in P1′ position in substrates cleaved by ADAM10 and 17. After searching for the least preferred amino acids in substrates of ADAM10 and 17, isoleucine was identified as never occurring in P1 of ADAM substrates, and a very low preference for aspartate or proline in P1′ and in P2′. To prove this, Peptides 4-6 were made. In these peptides the H-SI cleavage site was replaced with IPP, IPD, and IDP, respectively. While all 3 peptides were resistant to in vitro cleavage by ADAM17 (FIG. 5E), Peptides 5 and 6 were slowly cleaved by ADAM10 (data not shown). Peptide 4, with its IPP replacement for the H-SI cleavage site, appeared to be cleavage resistant in vitro.

### Example 6. TREM2 Ectodomain Shed from Cells is Cleaved Between H157 and S158

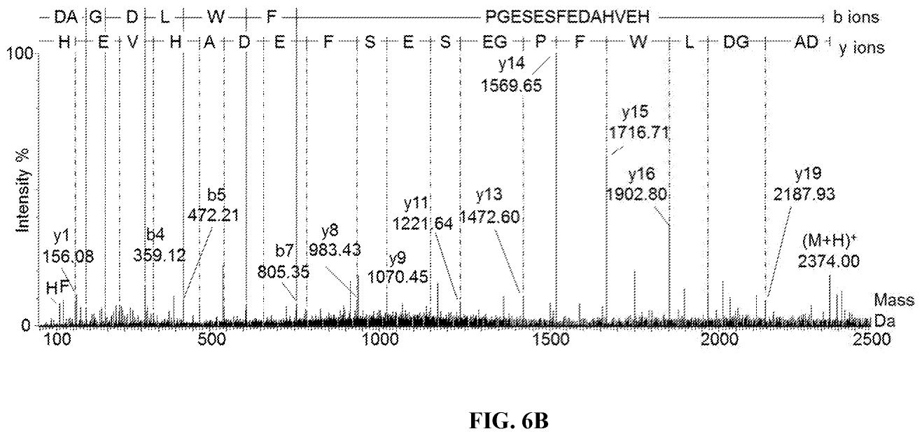

To substantiate the in vitro findings from the peptide analysis on sheddase cleavage site of TREM2 in a cellular system, HEK-FT cells were transiently transfected with hTREM2 or hTREM2-R47H in combination with hDAP12. Transfected cells from both conditions were treated with PMA or solvent. sTREM2 was immunopurified from cellular supernatant and subjected to trypsin or Asp-/Glu-C enzyme digestion followed by analysis of the peptides by LC-MS. In all four conditions the same N-terminal peptide was identified (D137-H157) indicating the main cleavage site between H157 and S158 (FIG. 6A-6B). These experiments show that neither PMA treatment nor the R47H mutation induce a shift of the main cleavage site.

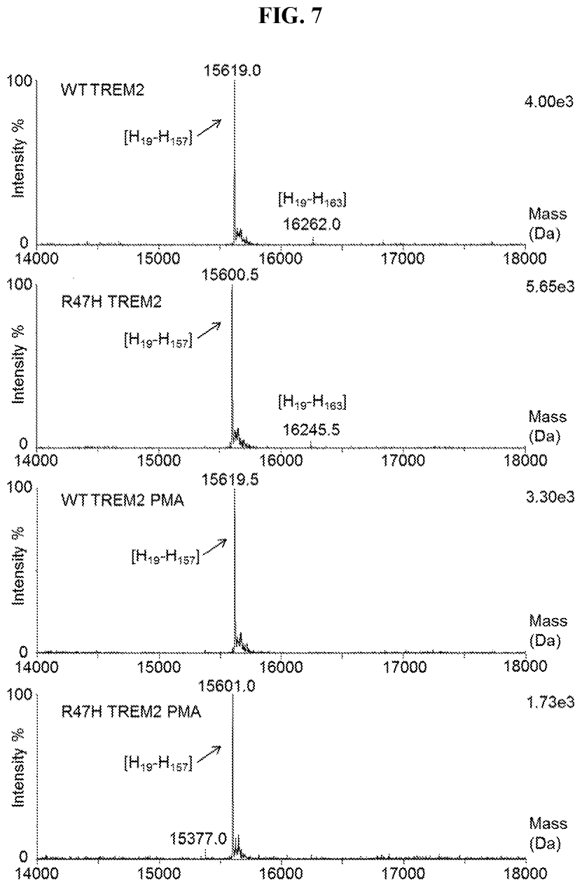

The next experiments were set out to identify the entire shed TREM2 ectodomain from the immunopurified cellular supernatant treated with PNGase-F and sialidase-A followed by LC-MS analysis. A 15,619 Dalton peptide species was detected in the supernatant from cells transfected with WT TREM2 which corresponds to de-glycosylated TREM219-157 (FIG. 7). A corresponding peptide of 15,600 Daltons was detected in the immunopurified cellular supernatant of R47H-TREM2 transfected cells. Due to the amino acids exchange this peptide is 19 Daltons lighter than the WT peptide and confirms the same cleavage site for mutated R47H-TREM2.

### Example 7. TREM2 is not O-glycosylated at Positions Close to the Cleavage Site

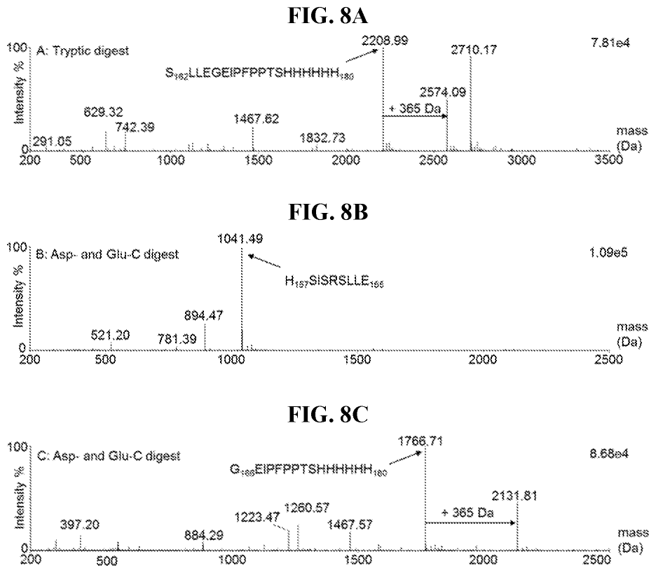

As post-translational modifications can effect sheddase activity (Goth et al., Proceedings of the National Academy of Sciences of the United States of America 112, 14623-14628, 2015; Schjoldager et al., Biochimica et Biophysica Acta 1820, 2079-2094, 2012), how glycosylation might effect TREM2 shedding was studied. Two putative O-glycosylation sites exist close to the identified H157 cleavage site of the TREM2 ectodomain: S160 and S168. Using C-terminally his-tagged hTREM2 and mapping glycosylation sites by mass spectrometry, it was shown that hTREM2 displays O-glycosylation at T171 and/or S172 (FIGS. 8A-8C), however, no O-glycosylation at S160 or S168 can be detected.

### Example 8. TREM2 Mutants with Mutations at the Sheddase Cleavage Site Show Increased Cell Surface Expression

The next set of experiments were aimed to substantiate in a cellular system that the TREM2 mutants with mutation at position 157-159 can reduce cleavage of TREM2 stalk region. The ADAM17 cleavage site amino acids HSI (position 157-159) within the stalk region of human TREM2 were replaced with amino acid IPD via site directed mutagenesis. The mutant construct was transiently expressed in HEK293-FT cells together with hDAP12 and cell surface expression of TREM2 was assessed as described in FIG. 2. Most interestingly, the TREM2 mutant with three amino acid replacements at the sheddase cleavage site showed a similar increase in TREM2 cell surface expression as the TREM2 mutant with truncation of the cleavage site (TRUNC3) or the TREM2 mutant with T2-double mutations (FIG. 4D), indicating resistance to sheddase cleavage in a cellular context.

The data presented here first investigated the contribution of ADAM10 and ADAM17 to shedding of TREM2. The pharmacological and genetic approaches used show unambiguously that ADAM17 is the pivotal protease contributing to this process. Enzyme selectivity of the ADAM inhibitors applied was determined in an in vitro cleavage assay and both inhibitors were used over a broad concentration range to investigate effects on TREM2 cell surface expression in CHO-hDAP12-hTREM2 cells and human M2A macrophages. To rule out that the pharmacological approach was not confounded by unspecific inhibition of other enzymes, these findings were corroborated by CRISPR/CAS9 knockout of ADAM10 or ADAM17 in the human monocytic THP-1 cell line. The knockout experiments confirmed that ADAM17 deficiency increased surface TREM2 and reduced sTREM2. The data presented here suggest that under steady state conditions TREM2 is cleaved mainly by ADAM17, but after activation of ADAMs by PMA, additional shedding mechanisms might come into play. Recent literature data indicated that under steady state conditions ADAM10 mediates generation of sTREM2 (Kleinberger, 2014). The main difference between that study and the results presented here is the use of HEK-Flp-In cells which lack co-expression of the signaling adaptor protein DAP12 together with TREM2. It might be that TREM2 after expression in a recombinant system in the absence of DAP12 has increased susceptibility to ADAM10 cleavage. Interestingly DAP12 has a 14 amino acids long extracellular domain (see Lanier & Bakker, Immunol Today 21, 611-614, 2000). The close proximity of the extracellular domain of DAP12 to the TREM2 cleavage site could suggest an interplay between the extracellular portions TREM2 and DAP12 that could regulate activation and shedding.

In addition to the investigation of constitutive shedding, PMA was used to enhance shedding of TREM2 from the cell surface. Shedding observed under these conditions in the ADAM17 deficient THP1 cells could be attributed to ADAM10 activity, but might also involve other mechanisms. For example, TREM2 ectodomain might be cleaved intracellularly during its transport from the ER to the plasma membrane, allowing for TREM2 secretion into the medium.

Recent studies have elucidated how PMA treatment and changes in sheddase activity are connected: PMA-triggered signaling cascades activate scramblases that enhance translocation of phosphatidylserine (PS) to the outer leaflet of the plasma membrane (Kodigepalli et al., Mol Cancer 12: 32, 2013). Here PS binds to a cationic motif in the membrane proximal domain of ADAM17 enabling the protease to execute its sheddase function (Sommer, Nature Communications 7, 11523, 2016). These experiments focused on ADAM17 and further studies are required to elucidate whether any of these findings extend to ADAM10, the closest homolog to ADAM17 within the ADAM family.

It is of note that PS has also been described as TREM2 ligand (Wang et al., Cell 160, 1061-1071, 2015; Cannon et al., Immunogenetics 64, 39-47, 2012; Daws, Journal of Immunology 171, 594-599, 2003; Song et al., Alzheimer's & Dementia 13: 381-387, 2017). Phosphatidylserine belongs to a range of membrane phospholipids which are exposed by damaged neurons and glial cells or released by damaged myelin. Further, negatively charged phospholipids like PS have been shown to associate with AP in lipid membranes (Ahyayauch et al., Biophys J 103, 453-463, 2012; Nagarathinam et al., J Neurosci 33, 19284-19294, 2013). In all these processes, PS acts as ligand for TREM2, and could at the same time facilitate its shedding.

The mutational analysis identified two amino acid stretches within the stalk region of TREM2 which are important for TREM2 shedding. The membrane distal area comprises the amino acids of the cleavage site. Interestingly, exchanging 5 amino acids close to the plasma membrane (mutant T2-WFR) also stabilized TREM2 at the cell surface. Although this has not been investigated for TREM2, co-clustering of ADAM17 with its substrate L-selectin (Schaff et al., Journal of Leukocyte Biology 83, 99-105, 2008) has been described and this part of the stalk region might contribute to this process enabling binding of ADAM17 to its substrate before cleavage is initiated upon activation of the proteolytic activity of ADAM17 (e.g., by PS).

Additional experiments identified the precise cleavage site for the TREM2 ectodomain. Three complementary approaches were applied, and all show the same cleavage site. First, peptides from the stalk region were subjected to in vitro cleavage with recombinantly expressed ADAM10 and ADAM17. Second, the C-terminus from tryptic peptides of the TREM2 ectodomain was purified from supernatant of HEK-FT cells recombinantly expressing hTREM2 and hDAP12 and determined. Third, the size of full length TREM2 ectodomain purified from cellular supernatant was verified. While the distance of the cleavage site from the plasma membrane (17 amino acids) is at the upper end when compared to most known ADAM17 substrates (12-16 amino acids, See Horiuchi, The Keio Journal of Medicine 62, 29-36, 2013; Overall & Blobel, Nature Reviews Molecular Cell Biology 8, 245-257, 2007), the sequence (P2:V, P1:H, P1′:S, P2′:I) is quite unique compared to known ADAM17 or ADAM10 substrates (Caescu et al., Biochem J 424, 79-88; Liu et al., Mol Immunol 62, 122-128, 2014; Tucher, 2014; Vahidi et al., Biochemical and Biophysical Research Communications 450, 782-787, 2014). ADAM10 and 17 have a preference for small hydrophobic residues at the P2 to P2′ positions, which drives substrate specificity. However, if cleavage sites for both ADAM17 and ADAM10 are compiled, (Tucher, 2014) arginine at P1 is quite common. Histidine at this position in TREM2 also carries a basic side chain with a positive charge. Exchanging P1, P1′ and P2′ with amino acids that are not common to the ADAM17/ADAM10 cleavage motif reduced cleavage. It is noteworthy that there was no shift of cleavage to another side within the peptide. This supports the observation that shedding seems to be confined to a region close to the plasma membrane and no shift of cleavage to a secondary site occurs. This is line with earlier findings suggesting that the position of the site relative to the transmembrane region and the first globular part of the protein is as important as the amino acids sequence of the cleavage site (Horiuchi, The Keio Journal of Medicine 62, 29-36, 2013; Hinkle, The Journal of Biological Chemistry 279, 24179-24188, 2004; Wang et al., The Journal of Biological Chemistry 277, 50510-50519, 2002).

The R47H variant confers a significantly increased risk to develop LOAD (Guerreiro, 2013; Jonsson, 2013). In contrast to some of the polymorphisms that confer increased risk for FTD like T66M (Guerreiro, 2013; Kleinberger, 2014; Borroni et al., Neurobiology of Aging 35, 934 e937-910, 2014; Le Ber, Neurobiology of Aging 35, 2419 e2423-2415, 2017) and Y38C (Guerreiro, 2013), which reduce cell surface expression of TREM2, the R47H mutation does not affect expression levels but rather functionally impairs TREM2 (Atagi, 2015; Bailey, 2015; Kleinberger, 2014; Wang, Cell 160, 1061-1071, 2015; Yin, 2016). Data presented here indicate that R47H mutation does not affect cleavage site, i.e. the size of soluble, shed TREM2 ectodomain. However, these experiments do not allow to conclude whether extent of shedding is changed, i.e. the amount of sTREM2 is altered.

Ectodomain shedding has been described to be influenced by O-glycosylation at serine or threonine residues which are within ±4 residues of the processing site (Goth, 2015; Schjoldager, 2012). The presence of O-glycosylation close to the cleavage site was investigated. The results indicate that within the stalk region TREM2 is only O-glycosylated at T171 and/or S172, but not at S168 or S160. Most likely this O-glycosylation site is too remote from the processing site to influence cleavage. These results are limited by the fact that the hTREM2 protein used for these studies was not shed from the cell surface but secreted through the secretory pathway in the expression system used for the production of the recombinant protein.

In the recent years a wealth of TREM2 variants have been identified that influence susceptibility for certain neurodegenerative diseases (See Dardiotis, Neurobiol Aging. 2017 May; 53:194.e13-194.e22). Most interestingly, one of these mutations H157Y (rs2234255, Ahyayauch, 2012; Cuyvers et al., Neurobiology of Aging 35, 726 e711-729, 2014; Jiang et al., Curr Neurovasc Res 13, 318-320, 2016; Ghani, Neurobiology of Aging 42, 217 e217-217 e213, 2016) is located precisely at the identified TREM2 cleavage site, and increases the propensity to develop AD. In vitro data show that ADAM10/17 has an increased preference for tyrosine in the P1′ position (Tucher, 2014). One could therefore speculate that this mutation may lead to enhanced constitutive shedding of TREM2 from the cell surface by ADAM10/17, and provide a mechanistic link between TREM2 shedding and development of AD, but exactly how this mutation affects shedding of TREM2 is currently unknown.

A fascinating question that arises from these results is whether sTREM2 has a physiological role. In initial phases of host defense or sterile inflammation, robust inflammation is advantageous for pathogen neutralization or removal of damaged tissue followed by the subsequent resolution response (Freire, Periodontology 2000, 63, 149-164). Upon resolution of inflammation, ADAM17 activity would diminish (Le Gall et al., Molecular Biology of the Cell 20, 1785-1794, 2009; Le Gall et al., Journal of Cell Science 123, 3913-3922, 2010) and the resulting increase in TREM2 would promote both resolution and phagocytosis. At the same time production of other pro-inflammatory cytokines like TNFα will become reduced.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A nucleic acid comprising a sequence encoding a human TREM2 mutant resistant to sheddase cleavage.

2. The nucleic acid of claim 1, wherein the sheddase is ADAM17 or ADAM10.

3. The nucleic acid of claim 1, wherein the sheddase is ADAM17.

4. The nucleic acid of claim 1, wherein the human TREM2 mutant comprises a stalk region comprising an amino acid sequence selected from any one of SEQ ID NOs: 33-40.

5. The nucleic acid of claim 1, wherein the human TREM2 mutant comprises a stalk region consisting of an amino acid sequence selected from any one of SEQ ID NOs: 33-40.

6. The nucleic acid of claim 1, wherein the human TREM2 mutant comprises a stalk region comprising an amino acid sequence selected from any one of SEQ ID NOs: 33-35.

7. The nucleic acid of claim 1, wherein the human TREM2 mutant comprises a stalk region consisting of an amino acid sequence selected from any one of SEQ ID NOs: 33-35.

8. The nucleic acid of claim 1, wherein the human TREM2 mutant comprises an amino acid sequence selected from any one of SEQ ID NOs: 41-48.

9. The nucleic acid of claim 1, wherein the human TREM2 mutant comprises an amino acid sequence selected from any one of SEQ ID NOs: 41-48.

10. The nucleic acid of claim 1, wherein the sequence comprises any one of SEQ ID NOs: 67-74.

11. The nucleic acid of claim 1 further comprising a promoter.

12. The nucleic acid of claim 11, wherein the promoter is a constitutive promoter.

13. The nucleic acid of claim 11, wherein the promoter is an inducible promoter.

14. The nucleic acid of claim 11, wherein the promoter is a synthetic promoter.

15. The nucleic acid of claim 11, wherein the promoter is a cell-type specific promoter.

16. The nucleic acid of claim 15, wherein the promoter drives the nucleic acid expression specifically in microglias, macrophages, or dendritic cells.

17. The nucleic acid of claim 11, wherein the promoter is selected from a TREM2 promoter, TMEM119 promoter, Hexb promoter, IBA1 promoter, CD45 promoter, CD11b promoter, Cst7 promoter, Lpl promoter, Csf1 promoter, Cs1R promoter, Itgax promoter, Clec7a promoter, Lilrb4 promoter, Tyrobp promoter, Ctsb promoter, Ctsd promoter, B2m promoter, Lyz2 promoter, Cx3cr1 promoter, Cst3 promoter, Ctss promoter, P2ryl2 promoter, C1qa promoter, or C1qb promoter.

18. The nucleic acid of claim 11, wherein the promoter is a TREM2 promoter.

19. The nucleic acid of claim 1 further comprising a polyadenylation signal.

20. The nucleic acid of claim 1 further comprising a second sequence encoding a DAP12 protein.

21. The nucleic acid of claim 20, wherein the DAP12 protein comprises SEQ ID NO: 49.

22. The nucleic acid of claim 20, wherein the DAP12 protein consists of SEQ ID NO: 49.

23. The nucleic acid of any of claims 20-22, wherein the nucleic acid comprises an internal ribosome entry site upstream of the second sequence.

24. The nucleic acid of any of claims 20-22, wherein the nucleic acid comprises a 2A sequence upstream of the second sequence, wherein the 2A sequence is selected from any one of SEQ ID NOs: 52-66.

25. A vector comprising the nucleic acid of any of claims 1-24.

26. The vector of claim 25, wherein the vector is selected from a DNA vector, an RNA vector, a plasmid, a cosmid, or a viral vector.

27. The vector of claim 26, wherein the viral vector is selected from a vector based on any one of the following viruses: lentivirus, adenovirus, adeno-associated virus (AAV), Herpes Simplex Virus (HSV), parvovirus, retrovirus, vaccinia virus, Sinbis virus, influenza virus, reovirus, Newcastle disease virus (NDV), measles virus, vesicular stomatitis virus (VSV), poliovirus, poxvirus, Seneca Valley virus, coxsackievirus, enterovirus, myxoma virus, or maraba virus.

28. The vector of claim 26, wherein the viral vector is a lentiviral vector.

29. The vector of claim 26, wherein the viral vector is an AAV vector.

30. The vector of claim 25 further comprising a selectable marker.

31. A cell comprising the nucleic acid of any of claims 1-24 or the vector of any of claims 25-30.

32. The cell of claim 31, wherein the cell is selected from a macrophage, a dendritic cell, or a microglia.

33. The cell of claim 31 or 32, wherein the cell expresses a detectable marker.

34. A polypeptide comprising an amino acid sequence selected from any one of SEQ ID NOs: 33-40.

35. The polypeptide of claim 34, wherein the polypeptide comprises an amino acid sequence selected from any one of SEQ ID NOs: 41-48.

36. A method of increasing TREM2 expression in a subject, the method comprising administering to the subject the nucleic acid of any of claims 1-24, the vector of any of claims 25-30, the cell of any of claims 31-33.

37. A method of treating a TREM2-related disease or disorder in a subject in need thereof, the method comprising administering to the subject the nucleic acid of any of claims 1-24, the vector of any of claims 25-30, the cell of any of claims 31-33.

38. The method of claim 36, wherein the subject has a TREM2-related disease or disorder.

39. The method of any of claims 36-38, wherein the subject is a human.

40. The method of any of claims 37-39, wherein the TREM2-related disease or disorder is a neuroinflammatory or neurodegenerative disease selected from Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, sphingomyelinlipidose (Niemann-Pick C), mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis.

41. The method of any of claims 37-39, wherein the TREM2-related disease or disorder is Alzheimer's disease.

42. The method of any of claims 37-39, wherein the TREM2-related disease or disorder is frontotemporal dementia.

43. The method of any of claims 36-42, wherein the nucleic acid, vector, or cell is administered to the subject through an intravenous, intracranial, intrathecal, subcutaneous, or intranasal route.

44. The method of any of claims 36-43, wherein the method further comprises administering a second agent to the subject.

45. The method of any of claims 36-44, wherein the method further comprises:
assaying the cell surface human TREM2 level in a sample obtained from a subject.

46. The method of claim 45, wherein the sample comprises cerebrospinal fluid.

47. The method of claim 45, wherein the cell surface human TREM2 level in a sample is determined by an assay selected from flow cytometry, immunohistochemistry, Western blotting, immunofluorescent assay, radioimmunoassay (MA), enzyme-linked immunosorbent assay (ELISA), homogeneous time resolved fluorescence (HTRF), or positron emission tomography (PET).

48. Use of the nucleic acid of any of claims 1-24, the vector of any of claims 25-30, the cell of any of claims 31-33, or the polypeptide of claim 34 or 35, for treatment of a TREM2-related disease or disorder in a subject.

49. Use of the nucleic acid of any of claims 1-24, the vector of any of claims 25-30, the cell of any of claims 31-33, or the polypeptide of claim 34 or 35, in the manufacture of a medicament for treatment of a TREM2-related disease or disorder in a subject.


SEQUENCE LISTING (89 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - DNA, 1076 bp, Homo sapiens
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaa

SEQ ID NO: 3 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 4 - protein, 222 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRPSQGSHLPSCLSKEPLGRRNPLPTHFHPSPPGLHLSHQDSSSQRPLGCSLAWTEARDTSTQ

SEQ ID NO: 5 - protein, 256 aa, Macaca fascicularis
MPDPLFSAVQGKDKILHKALCICPWPGKGGMEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPG

SEQ ID NO: 6 - protein, 227 aa, Mus musculus
MGPLHQFLLLLITALSQALNTTVLQGMAGQSLRVSCTYDALKHWGRRKAWCRQLGEEGPCQRVVSTHGVWLLAFLKKRNGSTVIADDTLAGTVTITLKNLQAGDAGLYQCQSLRGREAEVLQKVLVEVLEDPLDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 7 - protein, 62 aa, Homo sapiens
LHGSEADTLRKVLVEVLADPLDHRDAGDLWFPGESESFEDAHVEHSISRSLLEGEIPFPPTS

SEQ ID NO: 8 - protein, 57 aa, Homo sapiens
SLHGSEADTLRKVLVEVLADPLDHRDAGDLWFPGESESFEDAHVEHSISRAERHVKE

SEQ ID NO: 9 - protein, 60 aa, Homo sapiens
SLHGSEADTLRKVLVEVLADPLDHRDAGDLWFPGESESFEDAHVEHSISRPSQGSHLPSC

SEQ ID NO: 10 - protein, 62 aa, Macaca fascicularis
LHGSEADTLRKVLVEVLADPLDHRDAGDLWVPGESESFEDAHVEHSISRSLLEGEIPFPPTS

SEQ ID NO: 11 - protein, 59 aa, Mus musculus
LRGREAEVLQKVLVEVLEDPLDDQDAGDLWVPEESSSFEGAQVEHSTSRNQETSFPPTS

SEQ ID NO: 12 - protein, 41 aa, Homo sapiens
LWFPGESESFEDAHVEHSISRSLLEGEIPFPPTSILLLLAC

SEQ ID NO: 13 - protein, 25 aa, Artificial Sequence
  source: /note=“Description of Artificial Sequence Synthetic peptide”
LWFPGESESFEDAHVEHSILLLLAC

SEQ ID NO: 14 - protein, 35 aa, Artificial Sequence
  source: /note=“Description of Artificial Sequence Synthetic polypeptide”
LWFPGESESFEDAHVEHSISRSLLEGEIILLLLAC

SEQ ID NO: 15 - protein, 35 aa, Artificial Sequence
  source: /note=“Description of Artificial Sequence Synthetic polypeptide”
LWFPGESESFEDAHVEHSISRSLLEGTSILLLLAC

SEQ ID NO: 16 - protein, 35 aa, Artificial Sequence
  source: /note=“Description of Artificial Sequence Synthetic polypeptide”
LWFPGESESFEDAHVEHSISRSLFPPTSILLLLAC

SEQ ID NO: 17 - protein, 35 aa, Artificial Sequence
  source: /note=“Description of Artificial Sequence Synthetic polypeptide”
LWFPGESESFEDAHVEHSEGEIPFPPTSILLLLAC

SEQ ID NO: 18 - protein, 41 aa, Artificial Sequence
  source: /note=“Description of Artificial Sequence Synthetic polypeptide”
LWFPGESESFEDAHVYGGWGGWGPEGEIPFPPTSILLLLAC

SEQ ID NO: 19 - protein, 41 aa, Artificial Sequence
  source: /note=“Description of Artificial Sequence Synthetic polypeptide”
LWFPGESESFEDAHVEHSISRSLLEGEIWFRWTSILLLLAC

SEQ ID NO: 20 - protein, 41 aa, Artificial Sequence
  source: /note=“Description of Artificial Sequence Synthetic polypeptide”
LWFPGESESFEDAHVYGGWGGWGPEGEIWFRWTSILLLLAC

SEQ ID NO: 21 - protein, 41 aa, Artificial Sequence
  source: /note=“Description of Artificial Sequence Synthetic polypeptide”
LWFPGESESFEDAHVEIPDSRSLLEGEIPFPPTSILLLLAC

SEQ ID NO: 22 - protein, 41 aa, Artificial Sequence
  source: /note=“Description of Artificial Sequence Synthetic polypeptide”
LWFPGESESFEDAHVEIPPSRSLLEGEIPFPPTSILLLLAC

SEQ ID NO: 23 - protein, 41 aa, Artificial Sequence
  source: /note=“Description of Artificial Sequence Synthetic polypeptide”
LWFPGESESFEDAHVEIDPSRSLLEGEIPFPPTSILLLLAC

SEQ ID NO: 24 - protein, 60 aa, Homo sapiens
SLHGSEADTLRKVLVEVLADPLDHRDAGDLWFPGESESFEDAHVEHSISRSLLEGEIPFP

SEQ ID NO: 25 - protein, 20 aa, Artificial Sequence
  source: /note=“Description of Artificial Sequence Synthetic peptide”
SLHGSEADTLRKVLVEVLAD

SEQ ID NO: 26 - protein, 20 aa, Artificial Sequence
  source: /note=“Description of Artificial Sequence Synthetic peptide”
PLDHRDAGDLWFPGESESFE

SEQ ID NO: 27 - protein, 20 aa, Artificial Sequence
  source: /note=“Description of Artificial Sequence Synthetic peptide”
DAHVEHSISRSLLEGEIPFP

SEQ ID NO: 28 - protein, 14 aa, Artificial Sequence
  source: /note=“Description of Artificial Sequence Synthetic peptide”
VLVEVLADPLDHRD

SEQ ID NO: 29 - protein, 12 aa, Artificial Sequence
  source: /note=“Description of Artificial Sequence Synthetic peptide”
ESESFEDAHVEH

SEQ ID NO: 30 - protein, 20 aa, Artificial Sequence
  source: /note=“Description of Artificial Sequence Synthetic peptide”
DAHVEIPPSRSLLEGEIPFP

SEQ ID NO: 31 - protein, 20 aa, Artificial Sequence
  source: /note=“Description of Artificial Sequence Synthetic peptide”
DAHVEIPDSRSLLEGEIPFP

SEQ ID NO: 32 - protein, 20 aa, Artificial Sequence
  source: /note=“Description of Artificial Sequence Synthetic peptide”
DAHVEIDPSRSLLEGEIPFP

SEQ ID NO: 33 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVEIPDSRSLLEGEIPFPPTS

SEQ ID NO: 34 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVEIPPSRSLLEGEIPFPPTS

SEQ ID NO: 35 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVEIDPSRSLLEGEIPFPPTS

SEQ ID NO: 36 - protein, 47 aa, Artificial Sequence
  source: /note=“Description of Artificial Sequence Synthetic polypeptide”
SLHGSEADTLRKVLVEVLADPLDHRDAGDLWFPGESESFEDAHVEHS

SEQ ID NO: 37 - protein, 57 aa, Artificial Sequence
  source: /note=“Description of Artificial Sequence Synthetic polypeptide”
SLHGSEADTLRKVLVEVLADPLDHRDAGDLWFPGESESFEDAHVEHSEGEIPFPPTS

SEQ ID NO: 38 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVYGGWGGWGPEGEIPFPPTS

SEQ ID NO: 39 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVEHSISRSLLEGEIWFRWTS

SEQ ID NO: 40 - protein, 63 aa, Artificial Sequence
  source: /note=“Description of Artificial Sequence Synthetic polypeptide”
SLHGSEADTLRKVLVEVLADPLDHRDAGDLWFPGESESFEDAHVYGGWGGWGPEGEIWFRWTS

SEQ ID NO: 41 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEIPDSRSLLEGEIPFPPTSILLLLACIFLIKILAASALWAAAWHGQKPGTHPPSELDCGHDPGYQLQTLPGLRDT

SEQ ID NO: 42 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEIPPSRSLLEGEIPFPPTSILLLLACIFLIKILAASALWAAAWHGQKPGTHPPSELDCGHDPGYQLQTLPGLRDT

SEQ ID NO: 43 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEIDPSRSLLEGEIPFPPTSILLLLACIFLIKILAASALWAAAWHGQKPGTHPPSELDCGHDPGYQLQTLPGLRDT

SEQ ID NO: 44 - protein, 214 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSILLLLACIFLIKILAASALWAAAWHGQKPGTHPPSELDCGHDPGYQLQTLPGLRDT

SEQ ID NO: 45 - protein, 224 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSEGEIPFPPTSILLLLACIFLIKILAASALWAAAWHGQKPGTHPPSELDCGHDPGYQLQTLPGLRDT

SEQ ID NO: 46 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVYGGWGGWGPEGEIPFPPTSILLLLACIFLIKILAASALWAAAWHGQKPGTHPPSELDCGHDPGYQLQTLPGLRDT

SEQ ID NO: 47 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIWFRWTSILLLLACIFLIKILAASALWAAAWHGQKPGTHPPSELDCGHDPGYQLQTLPGLRDT

SEQ ID NO: 48 - protein, 230 aa, Artificial Sequence
  source: /note=“Description of Artificial Sequence Synthetic polypeptide”
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVYGGWGGWGPEGEIWFRWTSILLLLACIFLIKILAASALWAAAWHGQKPGTHPPSELDCGHDPGYQLQTLPGLRDT

SEQ ID NO: 49 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 50 - DNA, 608 bp, Homo sapiens
agacttcctccttcacttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaacaaaaaaaaaaaaaaaaaa

SEQ ID NO: 51 - protein, 14 aa, Artificial Sequence
  source: /note=“Description of Artificial Sequence Synthetic peptide”
SISRSLLEGEIPFP

SEQ ID NO: 52 - protein, 18 aa, Thosea asigna virus
EGRGSLLTCGDVEENPGP

SEQ ID NO: 53 - protein, 21 aa, Thosea asigna virus
GSGEGRGSLLTCGDVEENPGP

SEQ ID NO: 54 - protein, 19 aa, Porcine teschovirus-1
ATNFSLLKQAGDVEENPGP

SEQ ID NO: 55 - protein, 22 aa, Porcine teschovirus-1
GSGATNFSLLKQAGDVEENPGP

SEQ ID NO: 56 - protein, 20 aa, Equine rhinitis A virus
QCTNYALLKLAGDVESNPGP

SEQ ID NO: 57 - protein, 23 aa, Equine rhinitis A virus
GSGQCTNYALLKLAGDVESNPGP

SEQ ID NO: 58 - protein, 22 aa, Foot-and-mouth disease virus
VKQTLNFDLLKLAGDVESNPGP

SEQ ID NO: 59 - protein, 25 aa, Foot-and-mouth disease virus
GSGVKQTLNFDLLKLAGDVESNPGP

SEQ ID NO: 60 - protein, 25 aa, Strongylocentrotus purpuratus
DGFCILYLLLILLMRSGDVETNPGP

SEQ ID NO: 61 - protein, 20 aa, Amphimedon queenslandica
LLCFMLLLLLSGDVELNPGP

SEQ ID NO: 62 - protein, 20 aa, Amphimedon queenslandica
HHFMFLLLLLAGDIELNPGP

SEQ ID NO: 63 - protein, 20 aa, Saccoglossus kowalevskii
WFLVLLSFILSGDIEVNPGP

SEQ ID NO: 64 - protein, 20 aa, Branchiostoma floridae
KNCAMYMLLLSGDVETNPGP

SEQ ID NO: 65 - protein, 20 aa, Branchiostoma floridae
MVISQLMLKLAGDVEENPGP

SEQ ID NO: 66 - protein, 8 aa, Unknown
  source: /note=“Description of Unknown 2A consensus sequence”
  MOD_RES (2)..(2): Any amino acid
  MOD_RES (4)..(4): Any amino acid
DXEXNPGP

SEQ ID NO: 67 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaaattccggatagccgcagcctgctggaaggcgaaattccgtttccgccgaccagc

SEQ ID NO: 68 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaaattccgccgagccgcagcctgctggaaggcgaaattccgtttccgccgaccagc

SEQ ID NO: 69 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaaattgatccgagccgcagcctgctggaaggcgaaattccgtttccgccgaccagc

SEQ ID NO: 70 - DNA, 141 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaacatagc

SEQ ID NO: 71 - DNA, 171 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaacatagcgaaggcgaaattccgtttccgccgaccagc

SEQ ID NO: 72 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtgtatggcggctggggcggctggggcccggaaggcgaaattccgtttccgccgaccagc

SEQ ID NO: 73 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtggaacatagcattagccgcagcctgctggaaggcgaaatttggtttcgctggaccagc

SEQ ID NO: 74 - DNA, 189 bp, Artificial Sequence
  source: /note=“Description of Artificial Sequence Synthetic polynucleotide”
agcctgcatggcagcgaagcggataccctgcgcaaagtgctggtggaagtgctggcggatccgctggatcatcgcgatgcgggcgatctgtggtttccgggcgaaagcgaaagctttgaagatgcgcatgtgtatggcggctggggcggctggggcccggaaggcgaaatttggtttcgctggaccagc

SEQ ID NO: 75 - DNA, 20 bp, Artificial Sequence
  source: /note=“Description of Artificial Sequence Synthetic oligonucleotide”
gtaatgtgagagactttggg

SEQ ID NO: 76 - DNA, 20 bp, Artificial Sequence
  source: /note=“Description of Artificial Sequence Synthetic oligonucleotide”
ccgaagcccgggtcatccgg

SEQ ID NO: 77 - DNA, 25 bp, Artificial Sequence
  source: /note=“Description of Artificial Sequence Synthetic primer”
attagacaatacttactggggatcc

SEQ ID NO: 78 - DNA, 22 bp, Artificial Sequence
  source: /note=“Description of Artificial Sequence Synthetic primer”
ggaagctctggaggaatatgtg

SEQ ID NO: 79 - DNA, 20 bp, Artificial Sequence
  source: /note=“Description of Artificial Sequence Synthetic primer”
cccccaaacacctgatagac

SEQ ID NO: 80 - DNA, 20 bp, Artificial Sequence
  source: /note=“Description of Artificial Sequence Synthetic primer”
ccagagaggtggagtcggta

SEQ ID NO: 81 - DNA, 33 bp, Artificial Sequence
  source: /note=“Description of Artificial Sequence Synthetic oligonucleotide”
tcgtcggcagcgtcagatgtgtataagagacag

SEQ ID NO: 82 - DNA, 34 bp, Artificial Sequence
  source: /note=“Description of Artificial Sequence Synthetic oligonucleotide”
gtctcgtgggctcggagatgtgtataagagacag

SEQ ID NO: 83 - protein, 9 aa, Artificial Sequence
  source: /note=“Description of Artificial Sequence Synthetic peptide”
LLEGEIPFP

SEQ ID NO: 84 - protein, 12 aa, Artificial Sequence
  source: /note=“Description of Artificial Sequence Synthetic peptide”
SRSLLEGEIPFP

SEQ ID NO: 85 - protein, 21 aa, Artificial Sequence
  source: /note=“Description of Artificial Sequence Synthetic peptide”
DAGDLWFPGESESFEDAHVEH

SEQ ID NO: 86 - protein, 19 aa, Artificial Sequence
  source: /note=“Description of Artificial Sequence Synthetic peptide”
SLLEGEIPFPPTSHHHHHH

SEQ ID NO: 87 - protein, 9 aa, Artificial Sequence
  source: /note=“Description of Artificial Sequence Synthetic peptide”
HSISRSLLE

SEQ ID NO: 88 - protein, 15 aa, Artificial Sequence
  source: /note=“Description of Artificial Sequence Synthetic peptide”
GEIPFPPTSHHHHHH

SEQ ID NO: 89 - protein, 63 aa, Homo sapiens
SLHGSEADTLRKVLVEVLADPLDHRDAGDLWFPGESESFEDAHVEHSISRSLLEGEIPFPPTS